(19) United States
(12) Patent Application Publication
(10) Pub. No.: US 2022/0362320 A1
(43) Pub. Date: Nov. 17, 2022

(54) METHODS FOR PREPARING EXTRACTS FROM PLANTS AND FUNGI

(71) Applicant: Canna Stream Solutions Ltd., Edmonton (CA)

(72) Inventors: Behzad AHVAZI, Edmonton (CA); Usukuma Ekuere, Edmonton (CA); Krista Leicht, Edmonton (CA); Joseph John Vidmar, Edmonton (CA)

(73) Assignee: Canna Stream Solutions Ltd., Edmonton (CA)

(21) Appl. No.: 17/661,684

(22) Filed: May 2, 2022

Related U.S. Application Data
(60) Provisional application No. 63/183,110, filed on May 3, 2021.

(51) Int. Cl. A61K 36/185; A61K 31/352; A61K 31/05

(52) U.S. Cl. CPC A61K 36/185; A61K 31/352; A61K 31/05; A61K 2236/333; A61K 2236/331

(57) ABSTRACT

Extracts of a plant, fungus, waste, or part thereof, compositions comprising same, and methods of preparing and using same are provided. Extraction may be conducted using acetone/water extraction solution at predetermined ratios to yield extracts comprising desired phytocannabinoids, cannabimimetic compounds, terpenes, flavonoids, alkaloids, waxes, and other secondary metabolites. A composition comprises the extract and at least one acceptable carrier. A method of treating, preventing, or ameliorating a disease in a subject comprises administering to the subject an effective amount of the extract or composition.

### CROSS REFERENCE TO RELATED APPLICATIONS

The present application claims benefit of U.S. Provisional Patent Application No. 63/183,110 filed May 3, 2021, titled “Methods for Preparing Extracts from Plants and Fungi,” which is incorporated herein by reference in its entirety (where permitted).

### FIELD OF THE INVENTION

The present invention relates to extracts from plants, fungi, waste, or parts thereof, compositions comprising same, and methods of preparing and using same.

### BACKGROUND OF THE INVENTION

Plants have been a source of biologically active compounds for centuries and used extensively as crude material or pure compounds for therapeutic and recreational uses. As one of the world's oldest agricultural crops dating back six thousand years, cannabis is becoming a lucrative cash crop. Cannabis plants include basic anatomical structures including a main stem supporting leaf nodes from which fan leaves extend, and roots for anchoring and drawing nutrients into the plants below the surface of the growing medium. Male plants produce pollen sacs near the bases of the leaves to initiate seed production in female plants following pollination. However, seedless female plants produce flowers which are trimmed down into buds, which come together in a cola at the top of the stem. The buds are coated with glandular trichomes which produce and contain active chemical ingredients including, but not limited to, phytocannabinoids, aromatic oils (i.e., terpenes), and other compounds.

Among the phytocannabinoids, delta-9-tetrahydrocannabinol (“THC”) and cannabidiol (“CBD”) occur in the largest volume. THC causes psychoactive effects and is used to treat pain, nausea, insomnia, appetite loss, and depression. Being non-psychoactive, CBD is preferable to treat pain, nausea, inflammation, anxiety, epilepsy, and seizures. Other phytocannabinoids such as cannabichromene (“CBC”), cannabigerol (“CBG”), and cannabinol (“CBN”) exhibit similar therapeutic properties. Such phytocannabinoids may have potential applications for complex diseases including, but not limited to, cancer, multiple sclerosis, amyotrophic lateral sclerosis, Parkinson's disease, Alzheimer's disease, Crohn's disease, and post-traumatic stress disorder. Various terpenes include myrcene, pinene, limonene, caryophyllene, and linalool which may have potential in treating pain, inflammation, insomnia, anxiety, depression, and cancer.

Various mechanical separation and solvent-based extraction techniques have been developed to extract such medically valuable phytocannabinoids from the plant material. Mechanical separation techniques may include for example, shaking, sifting, dry sifting, grinding, sieving, pressing, extracting using ice water or dry ice, and applying heat and pressure to separate the trichomes from the plant, resulting in a concentrate such as kief, hash, or rosin.

Solvent-based extraction techniques utilize volatile chemicals including for example, hydrocarbons, carbon dioxide, and alcohols, to dissolve the trichomes from the plant material. As an example, butane and propane may be used to produce butane hash oil (BHO) and propane hash oil (PHO) extracts. Plant material is placed in a column with a screen at one end. Pressurizing and chilling the gas converts it into a liquid which passes through the column, extracting the phytocannabinoids and terpenes from the plant material. The solution is placed in a vacuum oven to evaporate the solvent, leaving behind the BHO or PHO. However, BHO and PHO extraction is potentially dangerous due to the flammability of butane and propane. In comparison, supercritical carbon dioxide oil extraction is a more environmentally-friendly technique of separating phytocannabinoids, waxes, and oils from the plant material. Supercritical carbon dioxide is heated and passed through the buds. The liquid passes through a separator to remove the carbon dioxide gas from the trichomes and terpenes. The gas is pushed through a condenser which allows the gas to liquefy and be recycled. However, carbon dioxide extraction requires expensive, specialized equipment in a closed loop system. In ethanol extraction, the plant material is soaked in ethanol for a predetermined time at any temperature, after which the plant material is removed, the solvent filtered, and the ethanol evaporated. Alternatively, hot alcohol solvent may be cycled through the flower to extract the phytocannabinoids and terpenes, but undesirably removes unwanted chlorophyll and waxes. Cold alcohol extraction avoids this problem, but can be difficult to scale up to large batches and post-processing requirements.

However, mechanical separation and solvent-based extraction techniques generate cannabis waste which requires proper management and disposal procedures, and is currently a complex issue for licensed producers. Cannabis waste may include, but is not limited to, wastes from cannabis flowers, trim and solid plant material used to create a cannabis isolate or concentrate; flammable solvents or chemicals used in the production of the cannabis isolate or concentrate; and discarded cannabis plant waste, spent solvents, and laboratory wastes from any cannabis processing or laboratory testing.

Liquid cannabis waste may be sent to a waste treatment facility. Solid cannabis waste may be made “unusable and unrecognizable” by grinding and mixing it with at least 50% non-cannabis material (e.g., cat litter, sand, plastic waste, sawdust, wood chips, cardboard waste, food waste, animal manure, grease or other compostable oil waste, leaf and yard waste) prior to disposal at an authorized landfill, compost facility, or anaerobic digester for processing. While such mixing renders solid cannabis waste unsuitable for consumption and propagation, the phytocannabinoids remain intact, which impairs potential secondary use of the cannabis waste for applications such as, for example, improving the growing of crops. In Canada, it is estimated that for every 1 kg of flower, there is 8 kg of waste; thus, the waste may exceed 7,200 mt. This untapped resource can be further divided into three waste components, namely leaves (2,160 mt); mature stems (3,240 mt); and roots (1,728 mt). The potential production of cannabinoids from the leaves and branches may be in the order of 20-60 mt.

Further, licensed producers are faced with tremendous costs to purchase sufficient non-cannabis material to mix with the same amount of cannabis waste to be destroyed, and to send double the amount of material to the landfill, compost facility, or anaerobic digester for processing. Incineration of cannabis waste is problematic since cannabis smoke or vapor may be released into the environment.

Accordingly, there is a need for improved methods of cannabis waste management which recycle, reuse, or repurpose cannabis waste by-products into value-added bioproducts for various applications, and reduce the costs of waste management for commercial cannabis producers. Such improved methods may also extend to plants other than cannabis, fungi, and other organisms.

### SUMMARY OF THE INVENTION

The present invention relates to extracts from plants, fungi, waste, or parts thereof, compositions comprising same, and methods of preparing and using same.

In one aspect, the invention comprises a method of preparing an extract of a plant, fungus, waste, or part thereof comprising contacting a powder obtained from the plant, fungus, waste, or part thereof with an extraction solution comprising acetone and water at a predetermined ratio, followed by filtration and solvent removal to obtain the extract comprising one or more phytocannabinoids, cannabimimetic compounds, terpenes, flavonoids, alkaloids, waxes, and other secondary metabolites.

In some embodiments, the predetermined ratio of acetone to water in the extraction solution ranges from 1:0 (v/v) to 5:1 (v/v). In some embodiments, the predetermined ratio of acetone to water in the extraction solution is selected from 1:0 (v/v), 1:2 (v/v), 1:1 (v/v), 3:2 (v/v), 2:1 (v/v), 4:1 (v/v), or 5:1 (v/v). In some embodiments, extraction is conducted for at least about 30 minutes to about 16 hours. In some embodiments, extraction is conducted for at least about 30 minutes to about 6 hours. In some embodiments, extraction is conducted at a temperature ranging from about −40° C. to about 60° C. In some embodiments, extraction is conducted at about room temperature. In some embodiments, extraction is conducted at about 50° C. In some embodiments, extraction is conducted at a pH ranging from 3.0 to 11.0. In some embodiments, extraction is conducted at a liquor to solid ratio ranging from 6:1 to 10:1. In some embodiments, extraction is conducted with sonication.

In some embodiments, the predetermined ratio of acetone to water in the extraction solution is 4:1 (v/v), and extraction is conducted at about 50° C. with sonication. In some embodiments, the predetermined ratio of acetone to water in the extraction solution is 4:1 (v/v), and extraction is conducted at a liquor to solid ratio of 10:1 for about 3 hours to about 6 hours.

In some embodiments, the one or more phytocannabinoids comprise delta-9-THC, delta-8-THC, iso-THC, THCA, CBD, CBDA, CBDV, CBDVa, CBC, CBCV, CBG, CBGV, CBGM, CBN, CBE, CBL, CBT, CBV, THCV, THCVA, THCC, and THCP.

In some embodiments, the one or more cannabimimetic compounds comprise alkyl amides, anandamide, guineensis, anthopogocyclolic acid, anthopogochromenic acid, and geranyl orsellinic acid.

In some embodiments, the plant, fungus, waste, or part thereof is selected from the genus Cannabis, Theobroma, Piper, Echinacea, Helichrysum, Rhododendron, Acmella, Rhodiola, Tuber, Copelandia, Gymnopilus, Inocybe, Panaeolus, Pholiotina, Pluteus, Psilocybe, or Hericium.

In some embodiments, the plant, waste, or part thereof is selected from the genus Cannabis, the predetermined ratio of acetone to water is selected from 1:2 (v/v), 1:1 (v/v), 3:2 (v/v), 2:1 (v/v), 4:1 (v/v), or 5:1 (v/v), and the one or more phytocannabinoids comprise delta-9-THC, delta-8-THC, iso-THC, THCA, CBD, CBDA, CBDV, CBDVa, CBC, CBCV, CBG, CBGV, CBGM, CBN, CBE, CBL, CBT, CBV, THCV, THCVA, THCC, and THCP.

In some embodiments, the plant, waste, or part thereof is selected from the genus Echinacea, the predetermined ratio of acetone to water is selected from 4:1 (v/v) to 5:1 (v/v), and the Echinacea extract comprises one or more cannabimimetic compounds, chicoric acid, echinacoside, and caftaric acid.

In some embodiments, the plant, waste, or part thereof is selected from the genus Rhodiola, the predetermined ratio of acetone to water is selected from 4:1 (v/v) to 5:1 (v/v), and the Rhodiola extract comprises one or more of salidroside, rosarin, and rosavin.

In some embodiments, the fungus, waste, or part thereof is selected from the genus Hericium, the predetermined ratio of acetone to water is selected from 4:1 (v/v) to 5:1 (v/v), and the Hericium extract comprises one or more glucans selected from alpha-glucan or beta-glucan.

In another aspect, the invention comprises an extract of a plant, fungus, waste, or part thereof obtained by the above method. In another aspect, the invention comprises a composition comprising the above extract and at least one acceptable carrier.

In another aspect, the invention comprises a method of treating, preventing, or ameliorating a disease or disorder in a subject, comprising administering to the subject an effective amount of the above extract or composition. In some embodiments, the disease or disorder comprises pain, nausea, insomnia, appetite loss, depression, inflammation, anxiety, epilepsy, seizures, cancer, multiple sclerosis, amyotrophic lateral sclerosis, Parkinson's disease, Alzheimer's disease, Crohn's disease, and post-traumatic stress disorder. In some embodiments, the invention comprises use of the above extract or composition to treat, prevent, or ameliorate a disease or disorder in a subject.

Additional aspects and advantages of the present invention will be apparent in view of the description, which follows. It should be understood, however, that the detailed description and the specific examples, while indicating preferred embodiments of the invention, are given by way of illustration only, since various changes and modifications within the scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 shows structures of cannabinoid standards used in Example 1.

FIG. 2 shows a representative total ion chromatogram (TIC) of the cannabinoid standards of FIG. 1.

FIG. 3 shows a calibration curve of cannabidiol (CBD) using peak area of [M+H]⁺.

FIG. 4 shows a calibration curve of cannabidiolic acid (CBDA) using peak area of [M+H]⁺.

FIG. 5 shows a calibration curve of tetrahydrocannabinol (THC) using peak area of [M+H]⁺.

FIG. 6 shows a calibration curve of tetrahydrocannabinolic acid (THCA) using peak area of [M+H]⁺.

FIG. 7 shows a calibration curve of cannabigerol (CBG) using peak area of [M+H]⁺.

FIG. 8 shows a calibration curve of cannabigerolic acid (CBGA) using peak area of [M+H]⁺.

FIG. 9 shows a calibration curve of cannabinol (CBN) using peak area of [M+H]⁺.

FIG. 10 shows a calibration curve of cannabichromene (CBC) using peak area of [M+H]⁺.

FIG. 11 shows a calibration curve of cannabidivarin (CBDV) using peak area of [M+H]⁺.

FIG. 12 shows a calibration curve of tetrahydrocannabivarin (THCV) using peak area of [M+H]⁺.

FIG. 13 shows a ¹H NMR spectrum of acetone/water (1:2) extract.

FIG. 14 shows a ¹H NMR spectrum of acetone/water (1:1) extract.

FIG. 15 shows a ¹H NMR spectrum of acetone/water (3:2) extract.

FIG. 16 shows a ¹H NMR spectrum of acetone/water (2:1) extract.

FIG. 17 shows a ¹H NMR spectrum of acetone/water (4:1) extract.

FIG. 18 shows a ¹H NMR spectrum of ethanol extract.

FIG. 19 shows A) representative TIC of acetone/water (1:2) extract; single ion monitoring for the accurate mass of B) CBD and THC; C) CBDA and THCA.

FIG. 20 shows A) representative TIC of acetone/water (1:1) extract; single ion monitoring for the accurate mass of B) CBD and THC; C) CBDA and THCA.

FIG. 21 shows A) representative TIC of acetone/water (3:2) extract; single ion monitoring for the accurate mass of B) CBD and THC; C) CBDA and THCA.

FIG. 22 shows A) representative TIC of acetone/water (2:1) extract; single ion monitoring for the accurate mass of B) CBD and THC; C) CBDA and THCA.

FIG. 23 shows A) representative TIC of acetone/water (4:1) extract; single ion monitoring for the accurate mass of B) CBD and THC; C) CBDA and THCA.

FIG. 24 shows A) representative TIC of ethanol extract; single ion monitoring for the accurate mass of B) CBD and THC; C) CBDA and THCA.

FIG. 25 shows a ¹H NMR spectrum of acetone/water (4:1) extract—cannabis sample extracted at 50° C.

FIG. 26 shows a ¹H NMR spectrum of acetone/water (4:1) extract—cannabis sample extracted at pH 11.0.

FIG. 27 shows a ¹H NMR spectrum of acetone/water (4:1) extract—cannabis sample extracted at pH 3.0.

FIG. 28 shows a ¹H NMR spectrum of acetone/water (4:1) extract—cannabis sample extracted with sonication.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It must be noted that as used herein and in the appended claims, the singular forms “a,” “an,” and “the” include plural referents unless the context clearly dictates otherwise. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

The present invention relates to extracts from plants, fungi, waste, or parts thereof, compositions comprising same, and methods of preparing and using same. As used herein, the term “extract” refers to a substance obtained by extracting raw plant, raw fungus, waste, or any part thereof using a solvent system. In some embodiments, the solvent system comprises a solution of acetone and water at predetermined ratios to separate desired active compounds from the plant, fungus, waste, or any part thereof, and to yield extracts comprising such active compounds.

In some embodiments, the extracts comprise one or more active compounds. As used herein, the term “active compound” means any isolate or concentrate capable of modifying or modulating the function of at least one given biological system. As used herein, the term “isolate” refers to one active compound. As used herein, the term “concentrate” refers to a combination of two or more active compounds. In some embodiments, the active compounds include, but are not limited to, phytocannabinoids, cannabimimetic compounds, terpenes, flavonoids, alkaloids, waxes, and other secondary metabolites.

In some embodiments, the plants comprise phytocannabinoid-containing plants. As used herein, the term “phytocannabinoid-containing plants” refers to plants capable of producing phytocannabinoids and/or cannabimimetic compounds including, but not limited to, those belonging to the families Cannabaceae, Malvaceae, Piperaceae, Asteraceae, Ericaceae, and Crassulaceae. In some embodiments, the plants include, but are not limited to, those belonging to the genera Cannabis, Theobroma, Piper, Echinacea, Helichrysum, Rhododendron, Acmella, and Rhodiola. In some embodiments, the plants include, but are not limited to, the species Cannabis sativa, Cannabis indica, Cannabis ruderalis, Theobroma cacao, Piper nigrum, Echinacea purpurea, Echinacea angustifolia, Helichrysum italicum, Helichrysum umbraculigerum, Rhododendron sinogrande, Acmella oleracea, and Rhodiola rosea.

While the methods are described herein as pertaining to plants, plant waste, or parts thereof, it will be appreciated by those skilled in the art that the methods may be applied also to non-plant matter including, but not limited to, fungi (for example, mushrooms and yeasts) which are capable of producing cannabimimetic compounds, alkaloids (for example, psilocybin and psilocin), and other secondary metabolites. As used herein, the term “cannabimimetic” refers to compounds which mimic the biological activity of cannabinoids. In some embodiments, the fungi include, but are not limited to, those belonging to the families Tuberaceae, Bolbitiaceae, Strophariaceae, Inocybaceae, Incertae sedis, Pluteaceae, Hymenogastraceae, and Hericiaceae. In some embodiments, the fungi include, but are not limited to, those belonging to the genera Tuber, Copelandia, Gymnopilus, Inocybe, Panaeolus, Pholiotina, Pluteus, Psilocybe, and Hericium.

As used herein, the term “phytocannabinoid” refers to any cannabinoid synthesized by a plant. As used herein, the term “cannabinoid” refers to a class of lipophilic molecules which interact with the human body's endocannabinoid system. The term includes, but is not limited to, delta-9-tetrahydrocannabinol (“delta-9-THC”); delta-8-tetrahydrocannabinol (“delta-8-THC”); iso-tetrahydrocannabinol (“iso-THC”); tetrahydrocannabinolic acid (“THCA”); cannabidiol (“CBD”); cannabidiolic acid (“CBDA”); cannabidivarin (“CBDV”); cannabidivarinic acid (“CBDVa”); cannabichromene (“CBC”); cannabichromevarin (“CBCV”); cannabigerol (“CBG”); cannabigerovarin (“CBGV”); cannabigerol monomethyl ether (“CBGM”); cannabinol (“CBN”); cannabielsoin (“CBE”); cannabicyclol (“CBL”); cannabicitran (“CBT”); cannabivarin (“CBV”); tetrahydrocannabivarin (“THCV”); tetrahydrocannabivaric acid (“THCVA”); tetrahydrocannabiorcol (“THCC”); and tetrahydrocannabiphorol (“THCP”). The term is also meant to include phytocannabinoids and cannabimimetic compounds which are not necessarily derived from Cannabis including, but not limited to, alkyl amides, anandamide, guineensis, anthopogocyclolic acid, anthopogochromenic acid, and geranyl orsellinic acid.

As used herein, the term “terpene” refers to a naturally occurring hydrocarbon having the general formula (C₅H₈)ₙ and derived biosynthetically from one or more molecules of isopentenyl pyrophosphate and/or dimethylallyl pyrophosphate. The term refers to terpenes produced by plants including, but not limited to, myrcene, pinene, limonene, caryophyllene, linalool, humulene, and nerolidol.

As used herein, the term “flavonoid” refers to a class of plant secondary metabolites, and can be classified into flavonoids or bioflavonoids; isoflavonoids, derived from 3-phenylchromen-4-one (3-phenyl-1,4-benzopyrone) structure; and neoflavonoids, derived from 4-phenylcoumarine (4-phenyl-1,2-benzopyrone) structure.

As used herein, the term “cannabis plant” refers to the genus of plants belonging to the family Cannabaceae including, but not limited to, wild type species and subspecies of Cannabis sativa, Cannabis indica, and Cannabis ruderalis, and variants thereof, including Cannabis chemovars (i.e., varieties characterized by virtue of chemical composition) and Cannabis plants which are the result of genetic crosses, self-crossed, or hybrids thereof. Suitable strains of Cannabis include, e.g., Indica-dominant (e.g., Blueberry, BC Bud, Holland's Hope, Kush, Northern Lights, Purple, and White Widow), Pure sativa (e.g., Acapulco Gold and Malawi Gold (Chamba), and Sativa-dominant (e.g., Charlotte's Web, Diesel, Haze, Jack Herer, Shaman, Skunk, Sour, and Te Puke Thunder). The term refers to the plant or any portion thereof including, but not limited to, flowers, fan leaves, sugar leaves, buds, branches, stems, seeds, roots, cotyledons, stipules, calyxes, pistils, stigmas, and trichomes. The term also refers to any active ingredients produced by the plant or portion thereof including, but not limited to, phytocannabinoids, terpenes, flavonoids, and other compounds.

As used herein, the term “leaf” or “leaves” refers to an organ of a vascular plant, as defined in botanical terms, and in particular, in plant morphology. In reference to Cannabis, the first pair of leaves usually have a single leaflet, the number gradually increasing up to a maximum of about thirteen leaflets per leaf (usually seven or nine), depending on variety and growing conditions. At the top of a flowering plant, this number again diminishes to a single leaflet per leaf. The lower leaf pairs usually occur in an opposite leaf arrangement and the upper leaf pairs in an alternate arrangement on the main stem of a mature plant. As used herein, the term “bud” refers to a flower-bearing stem or branch of the Cannabis plant, especially a stem or branch bearing a mass of female flowers with associated leaves. The stem or branch bearing the female flowers can be fresh, or can be dried. The pistils of the female Cannabis flower are surrounded by a mass of trichome-rich petals and leaves, and can contain higher concentrations of cannabinoids than do the plant leaves or stems. A bud, e.g., a mass of female flowers and associated leaves, usually covered with trichomes, can be further processed mechanically, i.e., “trimming” or “cleaning” the stem bearing the female flowers by removal of larger leaves and stem material. As used herein, “trichome” refers to a fine outgrowth or appendage on plants and certain protists. They are of diverse structure and function. Examples are hairs, glandular hairs, scales, and papillae. In reference to Cannabis, the trichome is a glandular trichome that occurs most abundantly on the floral calyxes and bracts of female plants.

As used herein, the term “biomass” refers to any organic material. As used herein, the term “waste biomass” refers to any organic waste from the plant, fungus, or portions thereof generated throughout harvesting, drying, curing, pruning, trimming, cloning, propagation, and other processing to produce an isolate or concentrate (e.g., flowers, leaves, stalks, roots, trim and solid plant material); and laboratory plant waste (e.g., goods/product samples or specimens remaining after laboratory testing).

Plant or fungal waste may relate not only to biomass. As used herein, the term “waste” generally refers to anything containing plant or fungal residuals or trace phytocannabinoids and/or cannabimimetic compounds including, but not limited to, spent waste from solvent (i.e., ethanol, butanol, hydrocarbons, carbon dioxide extraction) or non-solvent extraction; and defective, returned, expired, or non-compliant plant or fungal goods/products (e.g., flower, isolate, concentrate, solid/liquid edible, tincture, topical, vaporizer, and other consumable infused items).

It is desirable that the above-mentioned plant or fungal waste streams are managed in a manner that is both compliant and environmentally conscious. Accordingly, aspects of the present invention comprise methods of plant or fungal waste management which recycle, reuse, or repurpose plant or fungal waste into value-added bioproducts for various applications. The preparation of extracts obtained from plant or fungal waste biomass, determination of their properties, and formulations comprising the extracts are set forth herein.

In one aspect, the present invention comprises methods for preparing extracts from plant or fungal waste biomass. While the methods are described herein as pertaining to plant or fungal waste biomass, it will be appreciated by those skilled in the art that the methods may be applied also to non-waste materials such as, for example, the raw plant or raw fungus, or portions thereof.

In some embodiments, the extracts are obtained from the plant or fungal waste biomass as a starting material using the methods described herein. The methods generally involve at least the following sequential steps: contacting a powder obtained from the plant or fungal waste biomass with an extraction solution comprising acetone and water at a predetermined ratio, followed by filtration and solvent removal to obtain the extract comprising one or more phytocannabinoids, cannabimimetic compounds, terpenes, flavonoids, alkaloids, waxes, and other secondary metabolites. The physicochemical properties of the resultant extracts may be evaluated to assess their suitability for particular applications.

In order to prepare extracts, the plant or fungal waste biomass is first cleaned and dried. In some embodiments, the plant or fungal waste biomass is cleaned by washing or rinsing with a suitable cleaning agent. In some embodiments, the cleaning agent comprises an aqueous solvent. In some embodiments, the cleaning agent comprises water. The selected cleaning agent preferably is an inert solvent which does not react with or remove desired active ingredients from the plant or fungal waste biomass. The purpose of cleaning is to remove any debris including, but not limited to, dirt, dust, growth medium, insects, and other impurities, from the plant or fungal waste biomass.

The cleaned plant or fungal waste biomass is dried to remove the cleaning solution while retaining the dried biomass appearance and characteristics. Suitable drying techniques include, but are not limited to, air-drying, freeze-drying, lyophilization, liquid nitrogen, radiant energy vacuum dehydration (REV™, EnWave Corporation) and the like. The cleaned, dried biomass may be processed immediately or stored for future processing. The cleaned, dried biomass is subjected to size reduction including, but not limited to, chopping, grinding, grating, blending, cutting, pressing, crushing, disintegrating, pulverizing, milling, masticating, and the like. Size reduction is conducted to facilitate the extraction step which follows. In some embodiments, the cleaned, dried biomass is ground to a powder using a suitable grinder. Without being bound by any theory, the finer the particle size of the powder, the better result the extraction may achieve. The extraction efficiency may be enhanced by the small particle size due to the enhanced penetration of the acetone/water extraction solution and diffusion of solutes (i.e., the active compounds).

The powdered biomass is contacted with an extraction solution in order to yield extracts. In some embodiments, the extraction solution comprises an organic extraction solvent. In one embodiment, the organic extraction solvent is acetone. In some embodiments, the extraction solution comprises an organic extraction solvent and water. In some embodiments, the extraction solution comprises acetone alone, or acetone and water. In some embodiments, the ratio of acetone to water ranges from 1:0 (v/v) to 5:1 (v/v). In some embodiments, the ratio of acetone to water is 1:0 (v/v). In some embodiments, the ratio of acetone to water is 1:2 (v/v). In some embodiments, the ratio of acetone to water is 1:1 (v/v). In some embodiments, the ratio of acetone to water is 3:2 (v/v). In some embodiments, the ratio of acetone to water is 2:1 (v/v). In some embodiments, the ratio of acetone to water is 4:1 (v/v). In some embodiments, the ratio of acetone to water is 5:1 (v/v). In some embodiments, a co-solvent may be added to the solution of acetone and water including, but not limited to, ethanol, butanol, and the like.

In some embodiments, extraction is conducted with stirring. In some embodiments, extraction is conducted at a temperature ranging from about −40° C. to about 60° C. In some embodiments, extraction is conducted at room temperature. In some embodiments, extraction is conducted at about 50° C. In some embodiments, extraction is conducted at atmospheric pressure. In some embodiments, extraction is conducted at a pH ranging from 3.0 to 11.0. In some embodiments, extraction is conducted at a liquor to solid ratio ranging from 6:1 to 10:1. In some embodiments, extraction is conducted with sonication. In some embodiments, extraction is conducted for about 30 minutes to about 16 hours. In some embodiments, extraction is conducted for about 30 minutes to about 6 hours. In some embodiments, the ratio of acetone to water is 4:1 (v/v), and extraction is conducted at about 50° C. with sonication. In some embodiments, the ratio of acetone to water is 4:1 (v/v), and extraction is conducted at a liquor to solid ratio of 10:1 for about 3 hours to about 6 hours.

Following the extraction period, the resulting suspension comprises solid insoluble material and a liquid. The insoluble material is separated from the liquid by any suitable method including, but not limited to, filtration, dewatering screw press, and the like. In some embodiments, filtration is conducted by passing the suspension through a straining medium (for example, filter paper) having sufficient porosity to trap the insoluble material and allow the liquid or filtrate to pass therethrough. The filtrate is then dried to yield the extract. Suitable methods for drying include, but are not limited to, evaporation, vacuum drying, freeze drying, and the like. In some embodiments, the filtrate is dried using evaporation and vacuum drying. The filtrate is dried by evaporation under reduced pressure using a rotary evaporator, ensuring that the temperature does not exceed about 45° C. Following evaporation, the filtrate is then dried by vacuum drying to yield the final extract comprising the desired active compounds.

In some embodiments, the invention comprises extracts obtained by the methods described herein. In some embodiments, the methods decrease the risk of toxic residual chemicals in the extracts, and enable co-extraction of active compounds in the extracts. The physicochemical properties of the extracts may be evaluated to assess their suitability for particular applications. Suitable assays may include, but are not limited to, quantitative and qualitative analyses of active compounds therein, in vitro and in vivo pharmacological evaluations to assess activity, and the like.

In some embodiments, any evaluations are suitable, provided that they are focused upon indication of desired active compounds in either the extract or a representative sample from a batch of the extract in the event of large-scale manufacturing. In some embodiments, the active compounds comprise phytocannabinoids, cannabimimetic compounds, terpenes, flavonoids, alkaloids, waxes, and other secondary metabolites. In some embodiments, the phytocannabinoids comprise CBD, CBDA, THC, THCA, CBG, CBGA, CBC, CBN, CBDV, and THCV. In some embodiments, analysis of the extract may be determined by one or more techniques including, but not limited to, mass spectrometry (“MS”), gas chromatography (“GC”), liquid chromatography (“LC”), proton nuclear magnetic resonance spectroscopy (“proton NMR spectroscopy”), and gravimetric weight.

In some embodiments, Cannabis extracts are obtained by the methods described herein. In some embodiments, the Cannabis extract is obtained using the ratio of acetone to water selected from 1:2 (v/v), 1:1 (v/v), 3:2 (v/v), 2:1 (v/v), 4:1 (v/v), or 5:1 (v/v), and comprises one or more cannabinoids selected from delta-9-THC, delta-8-THC, iso-THC, THCA, CBD, CBDA, CBDV, CBDVa, CBC, CBCV, CBG, CBGV, CBGM, CBN, CBE, CBL, CBT, CBV, THCV, THCVA, THCC, and THCP. In some embodiments, the Cannabis extract is obtained using the ratio of acetone to water selected from 4:1 (v/v) to 5:1 (v/v).

In some embodiments, Echinacea extracts are obtained by the methods described herein. In some embodiments, the Echinacea extract is obtained using the ratio of acetone to water selected from 4:1 (v/v) to 5:1 (v/v), and comprises one or more cannabimimetic compounds, chicoric acid, echinacoside, and caftaric acid. In some embodiments, the one or more cannabimimetic compounds comprise alkyl amides which may have anti-inflammatory effects. Chicoric acid may have antioxidant, anti-cancer, anti-obesity, antiviral, and anti-diabetic effects. Echinacoside may have antioxidant and antibiotic effects, and inhibit apoptosis, demonstrating potential for use in neurological conditions. Caftaric acid may have antioxidant and anti-inflammatory effects.

In some embodiments, Rhodiola extracts are obtained by the methods described herein. In some embodiments, the Rhodiola extract is obtained using the ratio of acetone to water selected from 4:1 (v/v) to 5:1 (v/v), and comprises one or more of salidroside, rosarin, and rosavin. Salidroside may have anti-stress, adaptogenic, anxiolytic, and anti-depressive effects. Rosarin may have anti-inflammatory and neuroprotective effects. Rosavin may have anxiolytic, and anti-depressive effects.

In some embodiments, Hericium extracts are obtained by the methods described herein. In some embodiments, the extracts comprise Hericium erinaceus (also known as “Lion's Mane mushroom”) extracts. In some embodiments, the Hericium extract is obtained using the ratio of acetone to water selected from 4:1 (v/v) to 5:1 (v/v), and comprises glucans including, but not limited to, alpha-glucan and beta-glucan which may stimulate the immune system.

In some embodiments, the invention comprises formulations of the extracts obtained by the methods described herein. Powders of the extract may be used in that form directly as a loose powder or encapsulated powder. Alternatively, powders may be formulated into capsules, caplets, tablets and similar dosage forms. Further, powders may be formulated within liquid pervious membranes such as filters, meshes and the like, such as a tea bag-type infuser, for generating liquids containing the dissolved extract.

The powder form of the extract may be incorporated into liquids, formulated as solutions, dispersions or suspensions by dissolving the extract, for example as a drink, tincture, or drop. The extract may be administered alone, or with a carrier such as saline solution, an alcohol or water. An effective daily amount of the extract will vary with the subject, but will be less than is toxic while still providing a therapeutic effect. Solutions and formulations of the extract may lose some activity with aging and are thus either prepared in stable forms, or preferably prepared fresh for administration, for example in multicomponent kit form so as to avoid aging and to maximize the therapeutic effectiveness of the extract. Suitable kits or containers are well known for maintaining the phases of formulations separate until the time of use. A kit containing the extract in powder form may provide a sterile carrier such as water (and other ingredients) in a separate container in dosage specific amounts. The extract may be provided in a “tea bag”-type infuser or pouch, for generating liquid formulations at the time of use.

In some embodiments, the invention comprises compositions comprising the extract of the present invention in combination with one or more acceptable carriers (i.e., acceptable for pharmaceutical or recreational uses). As used herein, the term “carrier” means a suitable vehicle which is biocompatible and pharmaceutically or recreationally acceptable, including for instance, non-toxic liquid diluents and excipients which are suitable for administration. Those skilled in the art are familiar with any acceptable carrier that would be useful in this regard, and therefore the procedure for making compositions in accordance with the invention will not be discussed in detail (see for example Brunton et al., (eds) (2018) Goodman and Gilman's: The Pharmacological Bases of Therapeutics, 13^{th} Ed., McGraw-Hill Education. For standard dosages of conventional pharmacological agents, see, e.g., USP-NF 2021, Issue 1). Compositions may also include flavors, colorings, coatings, etc. All agents must be non-toxic and physiologically acceptable for the intended purpose, and must not substantially interfere with the activity of the extract so as to deleteriously affect the biological effect of the active compounds. Ingredients are thus only included in therapeutically acceptable amounts. It will be understood that extracts and compositions in the forms described above should be appropriately sterilized, purified and/or tested for microbiological parameters to ensure safety of administration. Extracts and compositions should be sealed in appropriate packaging or containers which for example, limit moisture (as in the case of powders or encapsulated powders) which could impair the biological activity of the extract.

The dosage of the extract depends upon many factors that are well known to those skilled in the art, for example, the particular form of the extract; the age, weight and clinical condition of the recipient patient; the concurrent therapeutic treatments; and the experience and judgement of the clinician or practitioner administering the therapy. A therapeutically effective amount of the extract provides either subjective relief of symptoms or an objectively identifiable improvement as noted by the clinician or other qualified observer. The extract may be administered through any suitable route of administration including, but not limited to, orally, nasally, dermally, osmotically, intraperitoneally, or intravenously at a dosage range and frequency (e.g., at least once daily) such that the level of active extract is maintained in the body. The dosage range varies with the route of administration, and the form and potency of the extract. Assessments following administration may include, but are not limited to, pharmacokinetics, biodistribution, uptake, biological response, toxicity, and the like.

Certain embodiments relate to the therapeutic use of the extracts in humans. In one aspect, the invention comprises a method of treating, preventing, or ameliorating a disease or disorder in a subject, comprising administering to the subject an effective amount of the extract or the composition. As used herein, the term “subject” means a human or other vertebrate. As used herein, the term “effective amount” means any amount of a formulation of the extract useful for treating, preventing, or ameliorating a disease or disorder upon administration. An effective amount of the composition provides either subjective relief of symptoms or an objectively identifiable improvement as noted by the clinician or other qualified observer. As used herein, the terms “treating,” “preventing” and “ameliorating” refer to interventions performed with the intention of alleviating the symptoms associated with, preventing the development of, or altering the pathology of a disease, disorder or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented. In some embodiments, the disease or disorder includes, but is not limited to, pain, nausea, insomnia, appetite loss, depression, inflammation, anxiety, epilepsy, seizures, cancer, multiple sclerosis, amyotrophic lateral sclerosis, Parkinson's disease, Alzheimer's disease, Crohn's disease, and post-traumatic stress disorder. In another aspect, the invention comprises use of the extract or composition to treat, prevent, or ameliorate a disease or disorder in a subject.

Embodiments of the present invention are described in the following Examples, which are set forth to aid in the understanding of the invention, and should not be construed to limit in any way the scope of the invention as defined in the claims which follow thereafter.

### Example 1—Extraction of Cannabinoids from Cannabis

Experiments were conducted to extract cannabinoids from commercial cannabis using an extraction solution of acetone and water prepared at different ratios and performing extraction at room temperature. Commercial cannabis was used in lieu of cannabis waste biomass for proof of concept. Multiple batches (3.5 g) of commercial cannabis were obtained from Nova Scotia Liquor Corporation (Penelope (Hybrid)), whole flower cannabis, containing 5.30 mg/g of THC (total THC 102 mg/g or 10.2%) and 2.10 mg/g CBD (total CBD 70.0 mg/g or 7.0%; Tweed Inc., Smiths Falls). The multiple batches of whole flower cannabis were combined and ground into a powder using a grinder. The powdered whole flower cannabis (about 10 g each) was extracted with acetone/water (100 mL) in triplicate. MilliQ™-water was added to the flask containing cannabis as per the ratios below with stirring for 10 minutes, and then acetone was added. The mixture was stirred at room temperature overnight. The extract was filtered through filter paper (Whatman Inc., Clifton, USA), and the filtrate was dried under reduced pressure using a rotary evaporator. The temperature did not exceed 45° C. during the evaporation process and the extract dried overnight under vacuum. The weight of the extract was measured and percentage extraction (%) was calculated using the following formula (I):

Percentage yield (%)=weight of extract/weight of cannabis×100 (I)

The acetone/water ratios used for extraction were 1:2 (v/v), 1:1 (v/v), 3:2 (v/v), 2:1 (v/v) and 4:1 (v/v). Ethanol was used as a standard. Table 1 sets out the percentage extraction yield of acetone/water and ethanol extracts. The average percentage extraction yield of the acetone/water extracts prepared from the powdered cannabis ranged from 13.66% to 35.36% (by dry weight). The percentage extraction yield was lowest using the acetone/water ratio of 1:2 (15.09±1.43) and highest for the acetone/water ratios of 2:1 (32.82±2.54) and 4:1 (33.19±1.49). The percentage extraction yield for the acetone/water ratios of 2:1 and 4:1 was greater than the percentage extraction yield for the ethanol standard (32.82±2.54).

Quantitative analysis of major cannabinoids was conducted by LC/MS using a Q-Exactive™ mass spectrometer equipped with electrospray ionization (Thermo Fisher Scientific, San Jose, Calif.). Solvents used for LC/MS were LCMS grade methanol (Fisher Chemicals, San Jose, Calif.). The MS parameters were sheath gas 50; auxiliary gas 10; spray voltage 3 kV; capillary temperature 300° C.; heater temperature 300° C.; data was acquired at 70,000 instrument resolution; column UPLC HSS-T3 column (Waters, 2.1×100 mm 1.8 μm); column temperature 30° C.; flow rate 0.4 mL/min; solvent gradient of 0.1% formic acid in water/0.1% formic acid in methanol with linear gradient (Table 2).

TABLE 1
Percentage extraction yield of Acetone/water and EtOH extracts

| Solvent | Sample wt | Extract wt | Extract | Extract Ave | |
|---|---|---|---|---|---|
| Ratio | (g) | (g) | (%) | (%) | SD |
| 1:2 | 10.05 | 1.65 | 16.42 | 15.09 | 1.43 |
| 1:2 | 10.02 | 1.53 | 15.27 | | |
| 1:2 | 10.02 | 1.36 | 13.57 | | |
| 1:1 | 10.03 | 2.52 | 25.12 | 22.27 | 2.47 |
| 1:1 | 10.03 | 2.09 | 20.84 | | |
| 1:1 | 10.02 | 2.09 | 20.86 | | |
| 3:2 | 10.04 | 2.24 | 22.31 | 25.80 | 3.31 |
| 3:2 | 10.00 | 2.62 | 26.20 | | |
| 3:2 | 10.00 | 2.83 | 28.50 | | |
| 2:1 | 10.04 | 3.29 | 32.77 | 32.82 | 2.54 |
| 2:1 | 10.03 | 3.04 | 30.31 | | |
| 2:1 | 10.03 | 3.55 | 35.39 | | |
| 4:1 | 10.03 | 3.42 | 34.1 | 33.19 | 1.49 |
| 4:1 | 10.03 | 3.41 | 34.00 | | |
| 4:1 | 10.01 | 3.15 | 31.47 | | |
| EtOH | 10.04 | 2.88 | 28.69 | 30.90 | 1.94 |
| EtOH | 10.01 | 3.17 | 31.67 | | |
| EtOH | 10.02 | 3.24 | 32.34 | | |

TABLE 2
Solvent gradients used for LC

| Time | 0.1% Formic acid | 0.1% Formic acid |
|---|---|---|
| (min) | in water (%) | in MeOH (%) |
| 0 | 20 | 80 |
| 2 | 16 | 84 |
| 6 | 14 | 86 |
| 7 | 0 | 100 |
| 9 | 0 | 100 |
| 9.5 | 20 | 80 |
| 12 | 20 | 80 |

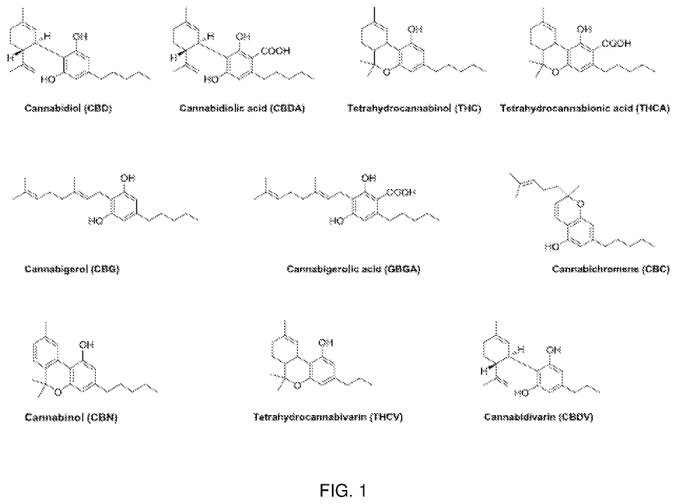

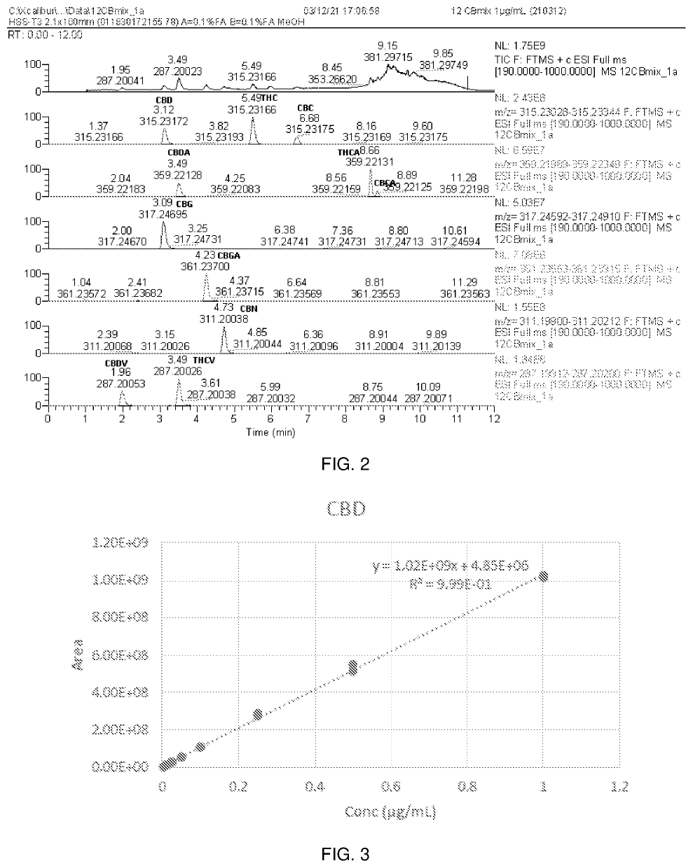

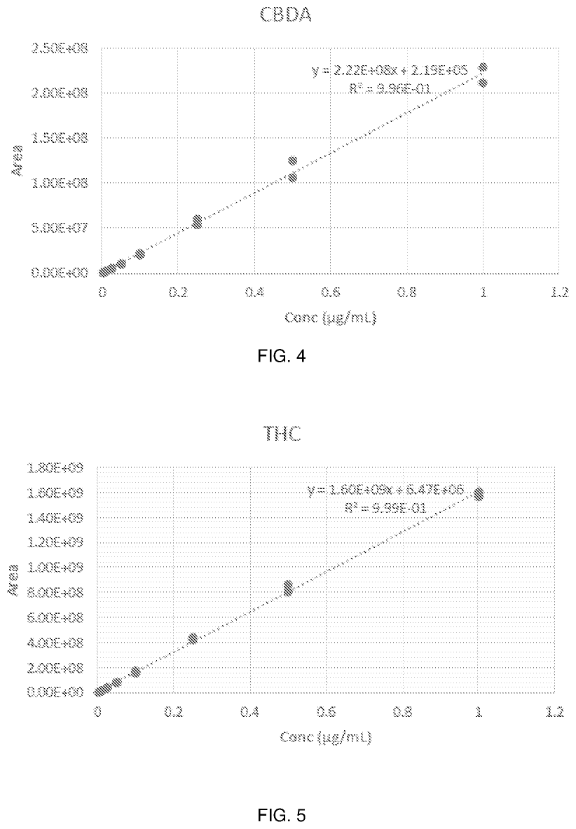

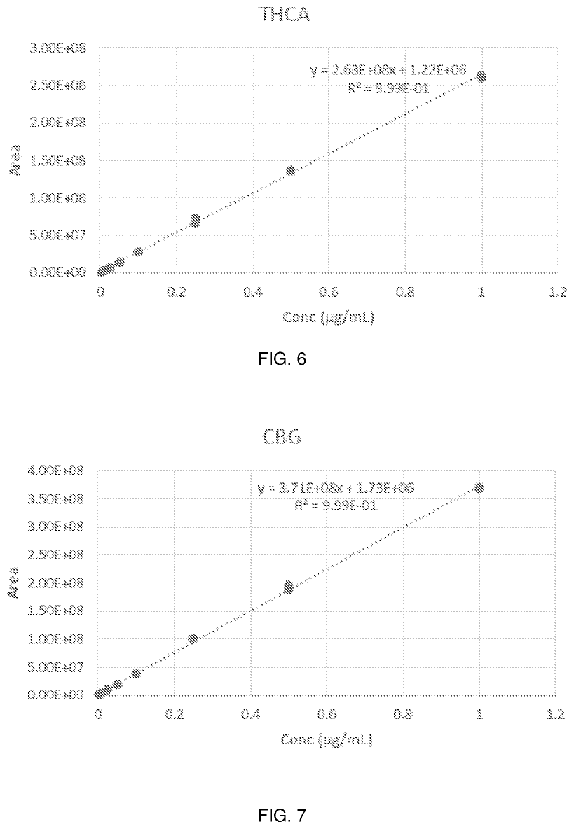

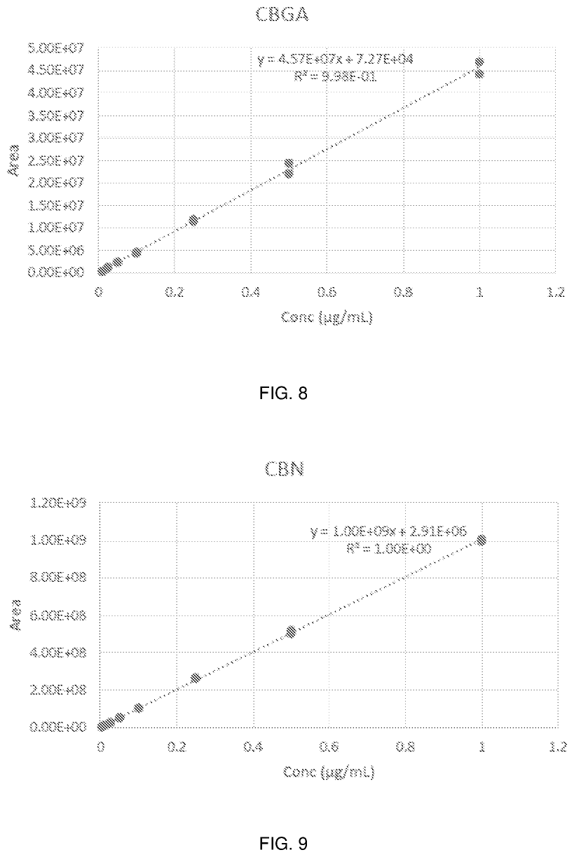

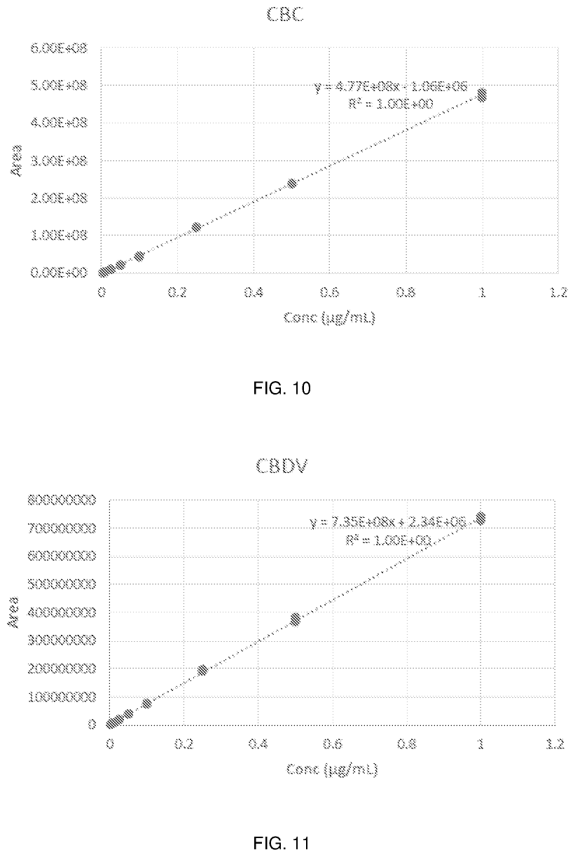

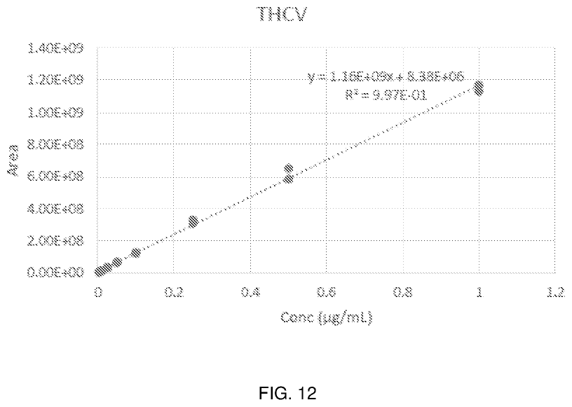

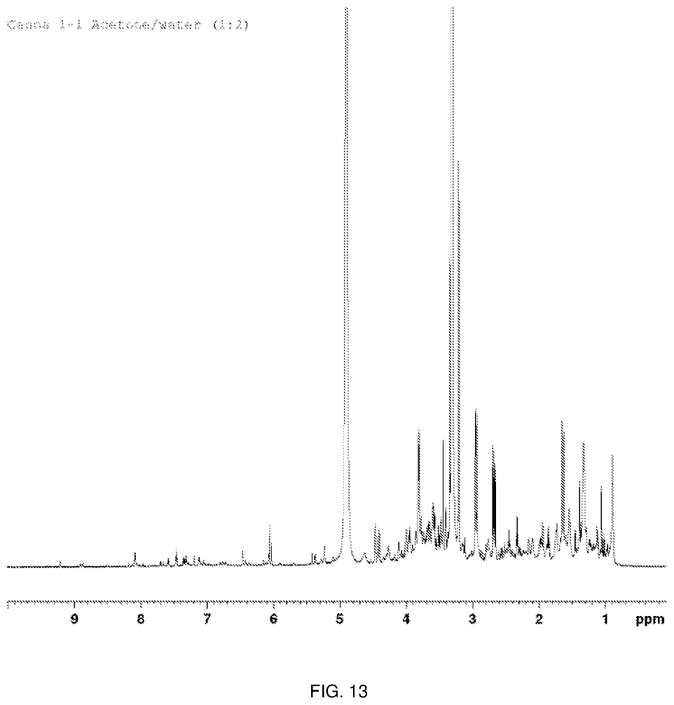

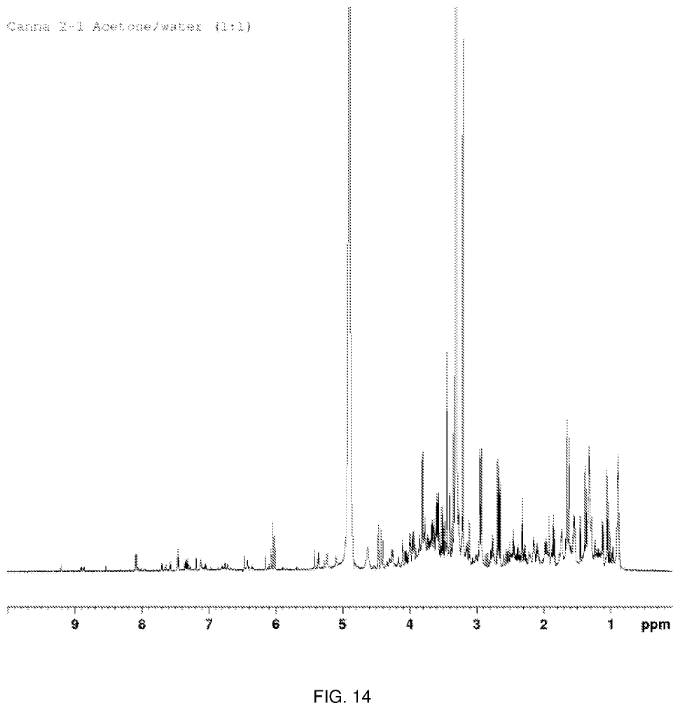

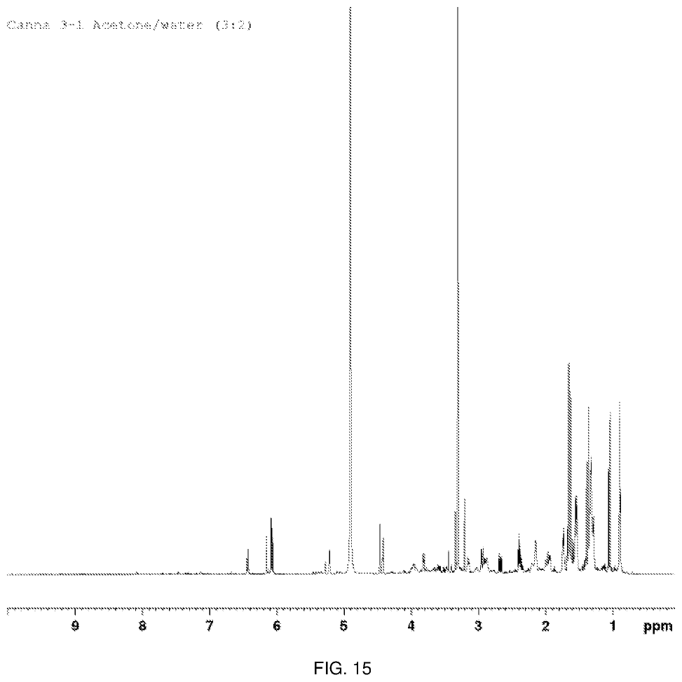

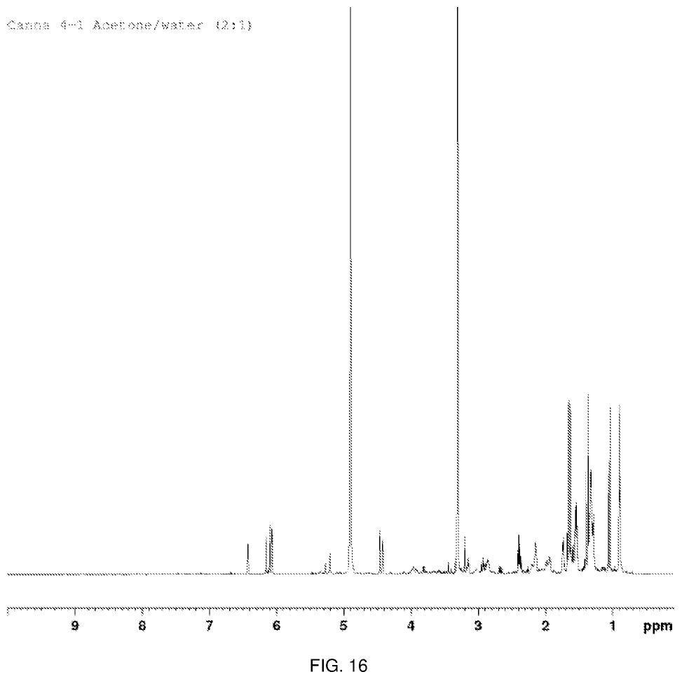

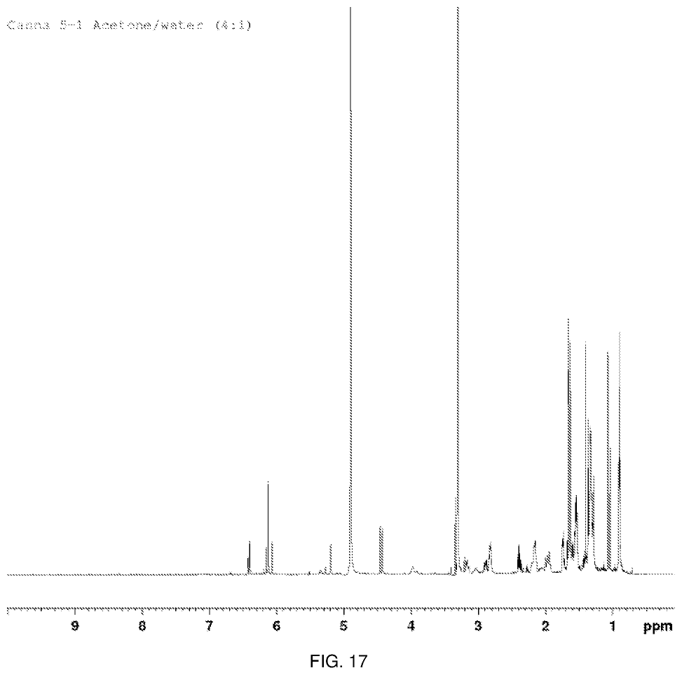

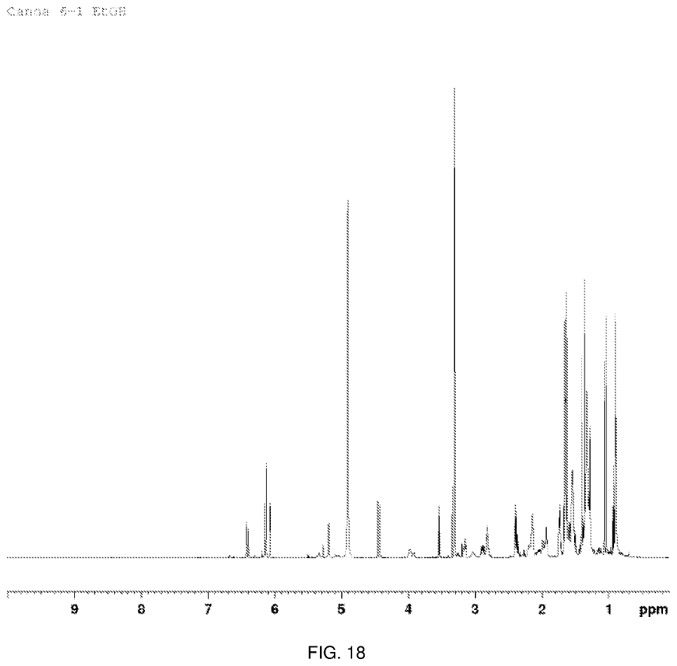

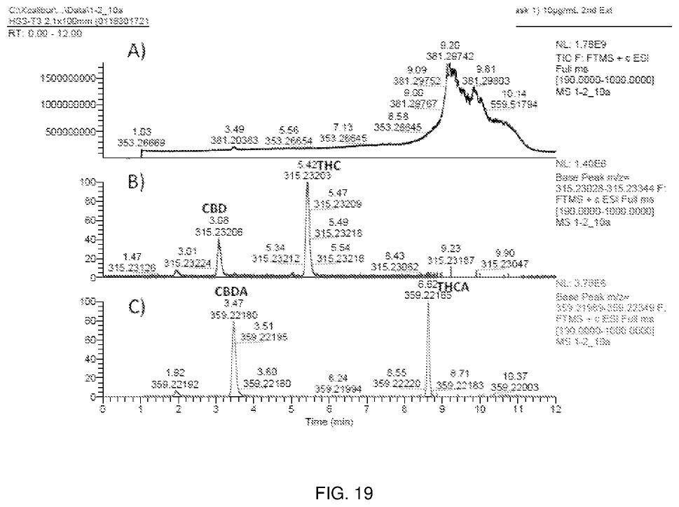

A calibration curve of standard cannabinoids was generated. The standard cannabinoids (Cerilliant, Round Rock, Tex.) are certified to 1 mg/mL, and included cannabidiol (CBD), cannabidiolic acid (CBDA), tetrahydrocannabinol (THC); tetrahydrocannabinolic acid (THCA), cannabigerol (CBG), cannabigerolic acid (CBGA), cannabichromene (CBC), cannabinol (CBN), cannabidivarin (CBDV), and tetrahydrocannabivarin (THCV). The structures of the standard cannabinoids are shown in FIG. 1. Standard cannabinoids were dissolved in methanol to prepare at various concentrations for a calibration curve (0.0025 μg/mL, 0.005 μg/mL, 0.01 μg/mL, 0.025 μg/mL, 0.05 μg/mL, 0.1 μg/mL, 0.25 μg/mL, 0.5 μg/mL, 1 μg/mL). Cannabinoids were eluted between 1.9 to 9.0 min and the representative total ion chromatogram (TIC) of the standards are shown in FIG. 2. Cannabinoids were ionized as [M+H]⁺ as well as sodium adduct [M+Na]⁺ and water loss ions. The [M+H]⁺ ion was used for quantification and the calibration curves of standard cannabinoids are shown in FIGS. 3-12.

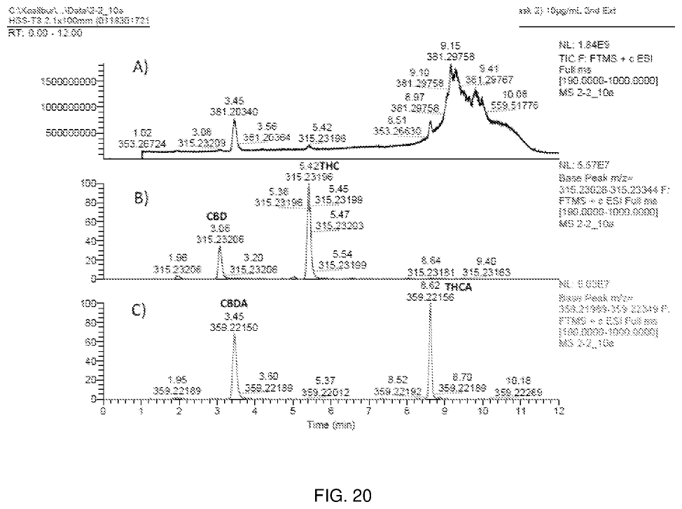

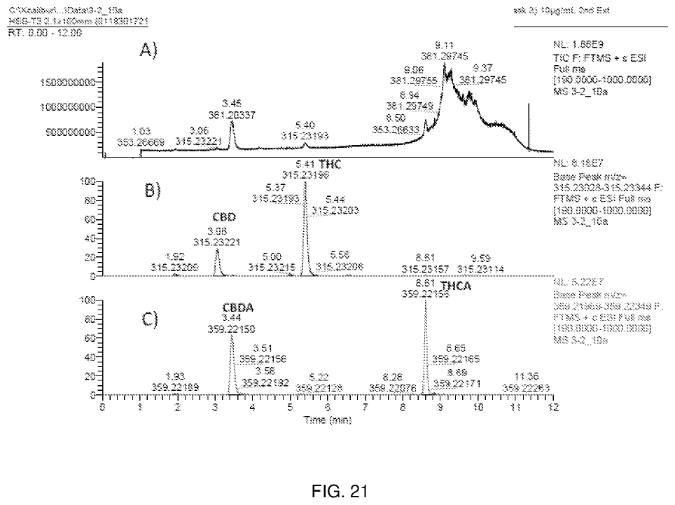

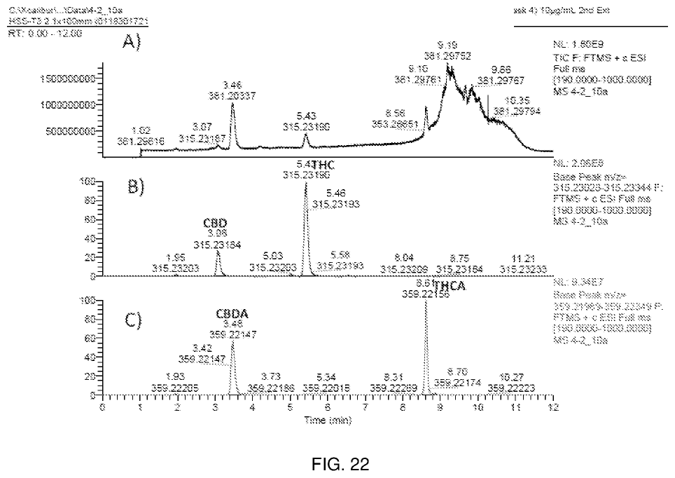

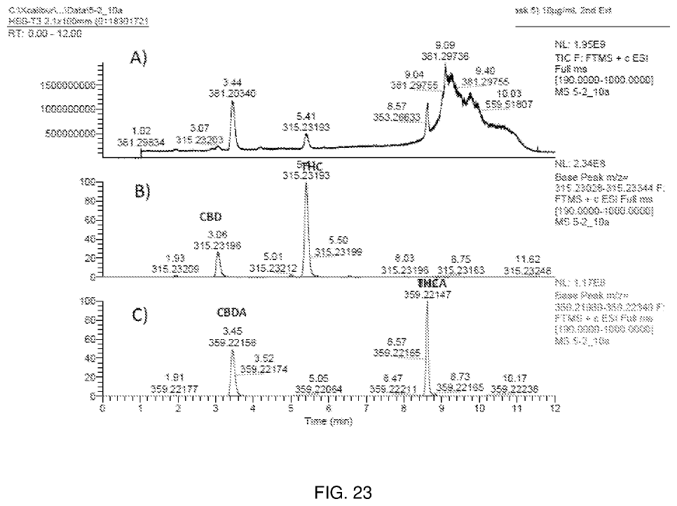

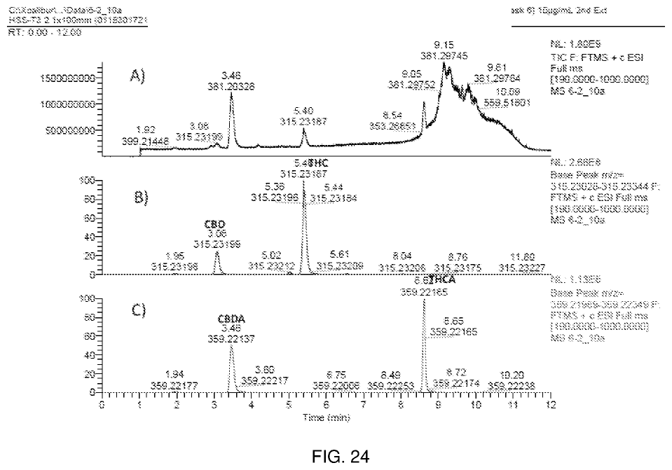

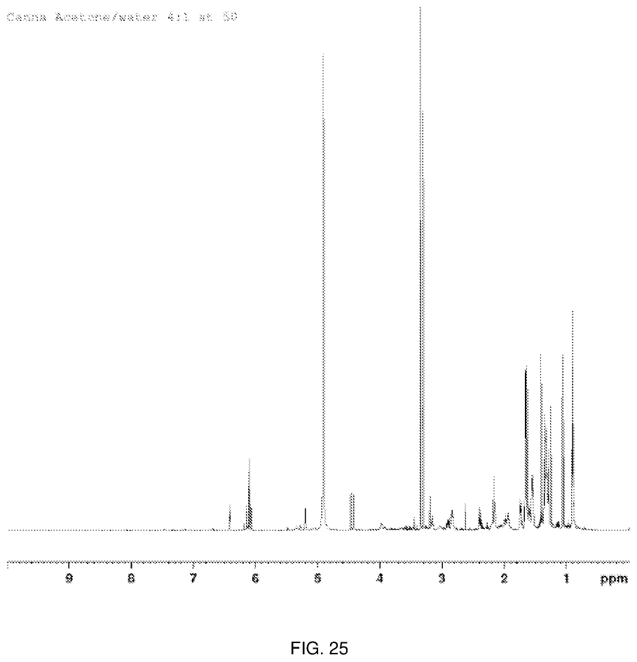

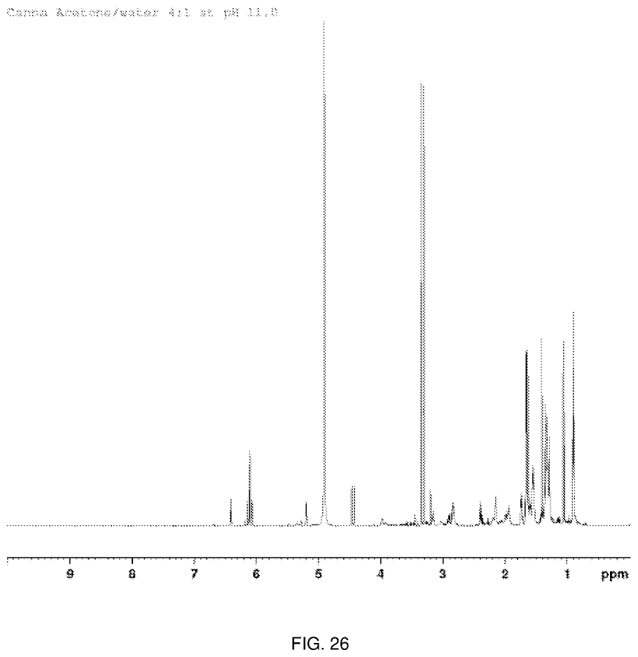

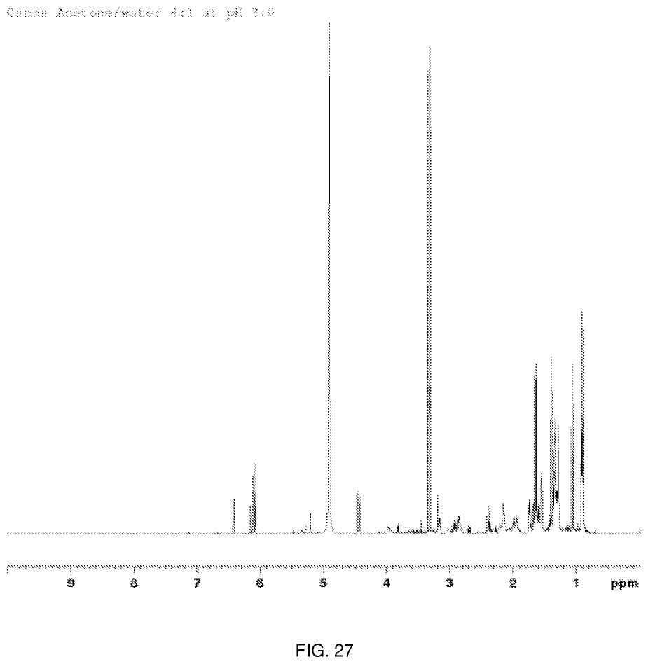

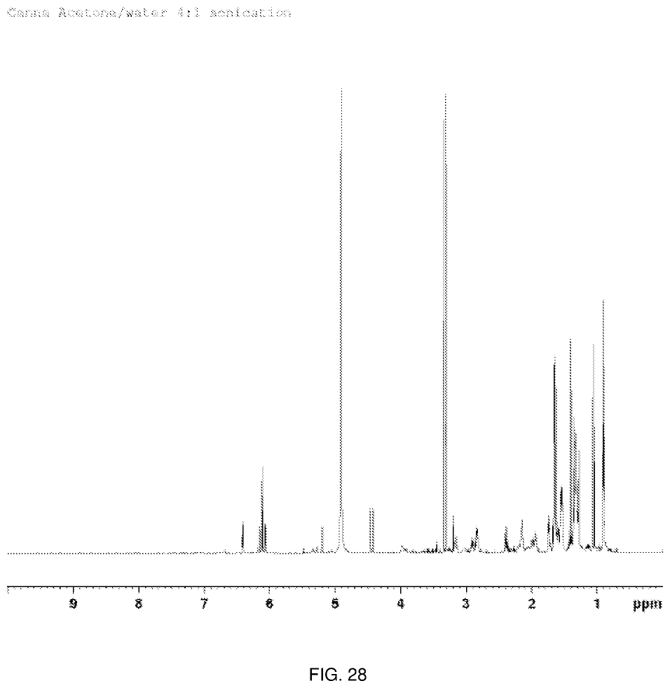

The concentration of individual cannabinoids in acetone/water extracts are summarized in Table 3. For each extract, 5 μL of 1 μg/mL and 10 μg/mL were injected for LC/MS analysis as the concentration of cannabinoids varied in the extracts. They were then converted back to 1 mg/mL extract concentrations in the values shown in Table 3. The representative TIC of acetone/water and ethanol extracts are shown in FIGS. 20-24.

All major cannabinoids (except for CBN, CBDV, THCV, CBGA, and CBC in some extracts) were detected in the acetone/water extracts regardless of the acetone/water ratio. The total cannabinoids were lowest using the acetone/water ratio of 1:2, considerably higher for the acetone/water ratios of 3:2 and 2:1, and highest for the acetone/water ratio of 4:1.

TABLE 3
Summary of major cannabinoid concentration in μg/mL
determined by LC/MS (average of duplicate injections)

| Acetone/ | | | | | | | | | | | Total |
|---|---|---|---|---|---|---|---|---|---|---|---|
| Water | CBD | THC | CBDA | THCA | CBG | CBN | CBDV | THCV | CBGA | CBC | Cannabinoids |
| 1:2 | 4.3 | 8.3 | 34.8 | 21.7 | 0.2 | — | — | — | 1.5 | — | 70.84 |
| 1:2 | 0.3 | 0.5 | 9.2 | 5.5 | — | — | — | — | — | — | 15.53 |
| 1:2 | 0.3 | 0.5 | 11.9 | 7.8 | — | — | — | — | — | — | 20.48 |
| 1:1 | 3.9 | 8.1 | 24.1 | 15.9 | 0.4 | — | — | — | 1.5 | — | 53.91 |
| 1:1 | 11.8 | 21.4 | 119.8 | 84.1 | 1.1 | 0.7 | — | — | 6.4 | 0.5 | 245.8 |
| 1:1 | 7.5 | 13.8 | 87.4 | 60.1 | 0.7 | 0.4 | — | — | 4.5 | — | 174.41 |
| 3:2 | 51.5 | 101.6 | 139.7 | 82.3 | 3.7 | 2.1 | — | 0.6 | 9.4 | 2.4 | 393.19 |
| 3:2 | 16.3 | 31.1 | 114.7 | 85.7 | 1.2 | 0.9 | — | — | 6.9 | 0.6 | 257.36 |
| 3:2 | 35.7 | 74.6 | 198.7 | 168.7 | 3.3 | 2.2 | — | 0.5 | 12.6 | 2 | 498.19 |
| 2:1 | 64.8 | 158.1 | 181.3 | 111.3 | 5.3 | 3.1 | — | 0.7 | 12.5 | 3.6 | 540.66 |
| 2:1 | 37 | 87 | 185.1 | 168.5 | 3.5 | 2.2 | — | 0.4 | 12.1 | 2.3 | 498.24 |
| 2:1 | 32.4 | 77.5 | 162.1 | 148.8 | 2.8 | 2 | — | 0.4 | 10.6 | 2 | 438.64 |
| 4:1 | 43.3 | 107 | 237 | 201 | 3.7 | 3.9 | — | 0.4 | 12 | 2.5 | 610.65 |
| 4:1 | 46.3 | 114.5 | 242.5 | 197.5 | 4 | 4.1 | — | 0.5 | 13.3 | 3 | 625.45 |
| 4:1 | 47.5 | 106 | 253.5 | 212 | 4 | 4.4 | — | 0.5 | 13.3 | 2.9 | 643.97 |
| EtOH | 73.3 | 190.2 | 234.8 | 178.6 | 6.2 | 3.6 | 0.3 | 0.8 | 16 | 4.1 | 707.83 |
| EtOH | 44.4 | 110.7 | 215.7 | 220.1 | 4.4 | 2.4 | — | 0.5 | 14.4 | 2.8 | 615.36 |
| EtOH | 41.8 | 103.9 | 218.2 | 224 | 3.8 | 2.3 | — | 0.5 | 14.5 | 2.7 | 611.7 |

The total cannabinoids concentration in the cannabis were calculated based on the extraction efficiency of acetone/water or ethanol and results are shown in Table 4. The average cannabinoids (mg/g) of the acetone/water extracts were lowest using the acetone/water ratio of 1:2 (5.59±5.23) and highest for the acetone/water ratios of 2:1 (161.14±14.04) and 4:1 (207.84±5.01).

TABLE 4
Total cannabinoid concentration in dry sample based
on the extract efficiency of acetone/water and EtOH.

| | | | Total | | | Average | |
|---|---|---|---|---|---|---|---|
| | Total | Extract | Cannabinoids | Dry | Cannabinoids | Cannabinoids | |
| Solvent | Cannabinoids | wt | in Ext | Sample | in Sample | in Sample | |
| Ratio | (μg/mL)* | (g)** | (mg) | (g)** | (mg/g) | (mg/g) | SD |
| 1:2 | 70.84 | 1.65 | 116.88 | 10.05 | 11.63 | 5.59 | 5.23 |
| 1:2 | 15.53 | 1.53 | 23.76 | 10.02 | 2.37 | | |
| 1:2 | 20.48 | 1.36 | 27.86 | 10.02 | 2.78 | | |
| 1:1 | 53.91 | 2.52 | 135.85 | 10.03 | 13.54 | 33.71 | 18.98 |
| 1:1 | 245.8 | 2.09 | 513.72 | 10.03 | 51.22 | | |
| 1:1 | 174.41 | 2.09 | 364.51 | 10.02 | 36.38 | | |
| 3:2 | 393.19 | 2.24 | 880.75 | 10.04 | 87.72 | 99.710 | 39.66 |
| 3:2 | 257.36 | 2.62 | 674.29 | 10.00 | 67.43 | | |
| 3:2 | 498.19 | 2.89 | 1439.77 | 10.00 | 143.98 | | |
| 2:1 | 540.66 | 3.29 | 1778.78 | 10.04 | 177.17 | 161.14 | 14.04 |
| 2:1 | 498.24 | 3.04 | 1514.66 | 10.03 | 151.01 | | |
| 2:1 | 438.64 | 3.55 | 1557.16 | 10.03 | 155.25 | | |
| 4:1 | 610.65 | 3.42 | 2088.42 | 10.03 | 208.22 | 207.84 | 5.01 |
| 4:1 | 625.45 | 3.41 | 2132.78 | 10.03 | 212.64 | | |
| 4:1 | 643.97 | 3.15 | 2028.5 | 10.01 | 202.65 | | |
| EtOH | 707.83 | 2.88 | 2038.54 | 10.04 | 203.04 | 198.57 | 4.14 |
| EtOH | 615.36 | 3.17 | 1950.68 | 10.01 | 194.87 | | |
| EtOH | 611.70 | 3.24 | 1981.91 | 10.02 | 197.80 | | |

*Total cannabinoids concentration was from Table 3 (μg/mL = μg/mg estract).
**Extract weight and dry sample weight were from Table 1.

The total CBD and THC concentration in the acetone/water extracts and their percentage in the cannabis sample are listed in Tables 5-7. As shown in Table 5, the average CBD (mg/g) in the acetone/water extracts were lowest using the acetone/water ratio of 1:2 (20.27±16.35) and highest using the acetone/water ratios of 2:1 (220.91±25.85) and 4:1 (290.02±10.42). As shown in Table 6, the average THC (mg/g) in the acetone/water extracts were lowest using the acetone/water ratio of 1:2 (14.78±13.28) and highest using the acetone/water ratios of 2:1 (250.44±25.00) and 4:1 (312.67±5.03). Table 7 shows the percentage total THC and CBD in the acetone/water extracts using the acetone/water ratio of 4:1 compared to those in the ethanol extract and the original cannabis batch (Tweed). While the percentage total THC were generally similar for all extracts and the original cannabis batch (Tweed Inc., Smiths Falls), the percentage total CBD appeared to be slightly higher in the acetone/water extracts (4:1) compared to the ethanol extract and the original cannabis batch (Tweed).

TABLE 5
Total CBD + CBDA concentration on acetone/water and EtOH extracts.

| | | | Total | | CBD + CBDA | |
|---|---|---|---|---|---|---|
| | | Extract | CBD + CBDA | CBD + CBDA | Average in | |
| Solvent | CBD + CBDA | wt | in Extract | in Extract | Extract | |
| Ratio | (μg/mg)* | (g)** | (mg) | (mg/g) | (mg/g) | SD |
| 1:2 | 39.1 | 1.65 | 64.49 | 39.090 | 20.27 | 16.35 |
| 1:2 | 9.50 | 1.53 | 14.55 | 9.5100 | | |
| 1:2 | 12.2 | 1.36 | 16.62 | 12.220 | | |
| 1:1 | 28.00 | 2.52 | 70.68 | 28.050 | 84.83 | 52.5 |
| 1:1 | 131.6 | 2.09 | 275.08 | 131.62 | | |
| 1:1 | 94.80 | 2.09 | 198.21 | 94.840 | | |
| 3:2 | 191.2 | 2.24 | 428.18 | 191.15 | 185.51 | 51.93 |
| 3:2 | 131.0 | 2.62 | 343.2 | 130.99 | | |
| 3:2 | 234.4 | 2.89 | 677.37 | 234.38 | | |
| 2:1 | 246.1 | 3.29 | 809.82 | 246.15 | 220.91 | 25.85 |
| 2:1 | 222.1 | 3.04 | 675.11 | 222.08 | | |
| 2:1 | 194.5 | 3.55 | 690.45 | 194.49 | | |
| 4:1 | 280.3 | 3.42 | 958.63 | 280.30 | 290.02 | 10.42 |
| 4:1 | 288.8 | 3.41 | 984.64 | 288.75 | | |
| 4:1 | 301.0 | 3.15 | 948.21 | 301.02 | | |
| EtOH | 308.1 | 2.88 | 887.39 | 308.12 | 276.09 | 27.74 |
| EtOH | 260.1 | 3.17 | 824.48 | 260.09 | | |
| EtOH | 260.1 | 3.24 | 842.61 | 260.07 | | |

*CBD and CBDA concentration were from Table 3 (μg/mL = μg/mg extract).
**Extract weight were from Table 1.

TABLE 6
Total THC + THCA concentration on acetone/water and EtOH extracts.

| | | | Total | | THC + THCA | |
|---|---|---|---|---|---|---|
| | | Extract | THC + THCA | THC + THCA | Average in | |
| Solvent | THC + THCA | wt | in Extract | in Extract | Extract | |
| Ratio | (μg/mg)* | (g)** | (mg) | (mg/g) | (mg/g) | SD |
| 1:2 | 30.1 | 1.65 | 49.6 | 30.06 | 14.78 | 13.28 |
| 1:2 | 6 | 1.53 | 9.2 | 6.02 | | |
| 1:2 | 8.3 | 1.36 | 11.23 | 8.26 | | |
| 1:1 | 24 | 2.52 | 60.54 | 24.02 | 67.84 | 41.11 |
| 1:1 | 105.6 | 2.09 | 220.62 | 105.56 | | |
| 1:1 | 73.9 | 2.09 | 154.5 | 73.92 | | |
| 3:2 | 183.9 | 2.24 | 411.84 | 183.86 | 181.27 | 63.29 |
| 3:2 | 116.7 | 2.62 | 305.82 | 116.73 | | |
| 3:2 | 243.2 | 2.89 | 702.95 | 243.24 | | |
| 2:1 | 269.4 | 3.29 | 886.43 | 269.43 | 250.44 | 22 |
| 2:1 | 255.6 | 3.04 | 776.89 | 255.56 | | |
| 2:1 | 226.3 | 3.55 | 803.49 | 226.34 | | |
| 4:1 | 308 | 3.42 | 1053.36 | 308.0 | 312.67 | 5.03 |
| 4:1 | 312 | 3.41 | 1063.92 | 312.0 | | |
| 4:1 | 318 | 3.15 | 1001.7 | 318.0 | | |
| EtOH | 368.9 | 2.88 | 1062.39 | 368.89 | 342.49 | 22.91 |
| EtOH | 330.7 | 3.17 | 1048.47 | 330.75 | | |
| EtOH | 327.8 | 3.24 | 1062.18 | 327.83 | | |

*THC and THCA concentration were from Table 3 (μg/mL = μg/mg extract).
**Extract weight were from Table 1.

TABLE 7
Percentage total THC and CBD on cannabis sample
based on acetone/water (4:1) and EtOH extracts.

| | | | | Average | | | Average |
|---|---|---|---|---|---|---|---|
| | | Total CBD | CBD in | CBD in | Total THC | THC in | THC in |
| Solvent | Sample | in Extract* | Sample | Sample | in Extract* | Sample | Sample |
| Ratio | (g) | (mg) | (%) | (%) | (mg) | (%) | (%) |
| 4:1 | 10.03 | 858.93 | 8.56 | 8.62 | 858.81 | 9.66 | 9.54 |
| 4:1 | 10.03 | 882.93 | 8.8 | | 981.08 | 9.78 | |
| 4:1 | 10.01 | 849.99 | 8.49 | | 919.56 | 9.19 | |
| EtOH | 10.04 | 804.22 | 8.01 | 7.65 | 999.11 | 9.95 | 9.76 |
| EtOH | 10.01 | 740.38 | 7.4 | | 962.66 | 9.62 | |
| EtOH | 10.02 | 755.63 | 7.54 | | 972.93 | 9.71 | |

| | | |
|---|---|---|
| Tweed - Percentage (%) CBD and THC in label | 7 | 10.2 |

*Cannabis sample weight/extract weight were from Table 1 and cannabinoids (THC, THCA, CBD & CBDA) concentrations were from Table 3. Total CBD and total THC concentration were calculated using following formula Total THC = THC + (THCA × 0.877) and Total CBD = CBD + (CBDA × 0.877)

In summary, the above results suggest that a mixture of acetone/water as a medium is capable of extracting cannabinoids efficiently. The optimum ratio mixture is found to be 4:1 (v/v). The extent of extracted cannabinoids of acetone/water is comparable to ethanol.

### Example 2—Effects of Temperature, Sonication, and pH on Extraction of Cannabinoids from Cannabis or Cannabis Waste

Achieving greater cannabinoid extraction efficiency was further evaluated by using temperature higher than ambient (>25° C.), ultrasonication, and different pH conditions. Heating acetone/water (4:1) at 50° C. and sonication improved the cannabinoid extraction.

TABLE 8
Percentage extraction yield of Acetone/water under various conditions:
at 50° C., pH 3 and 11, and with aid of sonication.

| Solvent | | Sample wt | Extract wt | Extract |
|---|---|---|---|---|
| Ratio | Condition | (g) | (g) | (%) |
| 4:1 | 50° C. | 7.00 | 2.70 | 38.57 |
| 4:1 | pH 11 | 7.00 | 2.41 | 34.43 |
| 4:1 | pH 3 | 7.01 | 2.40 | 34.24 |
| 4:1 | Sonication | 6.05 | 2.12 | 35.04 |

TABLE 9
Summary of major cannabinoids concentration in μg/mL determined by LC/MS (average of duplicate
injections) under various conditions: at 50° C., pH 3 and 11, and with aid of sonication.

| Solvent | | | | | | | | | | | Total |
|---|---|---|---|---|---|---|---|---|---|---|---|
| Ratio | Condition | CBD | THC | CBDA | THCA | CBG | CBN | THCV | CBGA | CBC | Cannabinoids |
| 4:1 | 50° C. | 55.9 | 122.9 | 226.1 | 196 | 5.0 | 3.3 | 0.5 | 14.1 | 2.8 | 627.01 |
| 4:1 | pH 11 | 39.7 | 87.5 | 192.9 | 181 | 3.8 | 2.6 | 0.4 | 11.8 | 2.0 | 522.06 |
| 4:1 | pH 3 | 42.0 | 98.4 | 220.2 | 203 | 4.0 | 2.9 | 0.4 | 12.9 | 2.6 | 586.55 |
| 4:1 | Sonication | 48.0 | 109.9 | 247.1 | 225 | 4.6 | 3.2 | 0.4 | 14.9 | 2.6 | 655.87 |

TABLE 10
Total cannabinoids concentration in dry sample based on the extraction
efficiency of acetone/water (4:1) under various conditions:
at 50° C., pH 3 and 11, and with aid of sonication.

| | | | | Total | | |
|---|---|---|---|---|---|---|
| | | Total | Extract | Cannabinoids | Dry | Cannabinoids |
| Solvent | | Cannabinoids | wt | in Ext | Sample | in Sample |
| Ratio | Condition | (μg/mL)* | (g)** | (mg) | (g)** | (mg/g) |
| 4:1 | 50° C. | 627.0 | 2.70 | 1692.92 | 7.00 | 241.85 |
| 4:1 | pH 11 | 522.1 | 2.41 | 1258.16 | 7.00 | 179.74 |
| 4:1 | pH 3 | 586.6 | 2.40 | 1407.72 | 7.01 | 200.82 |
| 4:1 | Sonication | 655.9 | 2.12 | 1390.44 | 6.05 | 229.82 |

*Total cannabinoids concentration were from Table 9 (μg/mL = μg/mg extract).
**Extract weight and dry sample weight were from Table 8.

TABLE 11
Total CBD + CBDA concentration on acetone/water
(4:1) extracts under various conditions: at 50°
C., pH 3 and 11, and with aid of sonication.

| | | | | Total | |
|---|---|---|---|---|---|
| | | | | CBD + | CBD + |
| | | CBD + | Extract | CBDA | CBDA |
| Solvent | Extra | CBDA | wt | in Extract | in Extract |
| Ratio | Condition | (μg/mg)* | (g)** | (mg) | (mg/g) |
| 4:1 | 50° C. | 282.01 | 2.7 | 761.42 | 282.01 |
| 4:1 | pH 11 | 232.6 | 2.41 | 560.58 | 232.6 |
| 4:1 | pH 3 | 262.25 | 2.4 | 629.43 | 262.26 |
| 4:1 | Sonication | 295.12 | 2.12 | 625.65 | 295.12 |

*CBD + CBDA concentration were from Table 9 (μg/mL = μg/mg extract).
**Extract weight was from Table 8.

TABLE 12
Total THC + THCA concentration on acetone/water
(4:1) extracts under various conditions: at 50°
C., pH 3 and 11_{;} and with aid of sonication.

| | | | | Total | |
|---|---|---|---|---|---|
| | | | | THC + | THC + |
| | | THC + | Extract | THCA | THCA |
| Solvent | Extra | THCA | wt | in Extract | in Extract |
| Ratio | Condition | (μg/mg)* | (g)** | (mg) | (mg/g) |
| 4:1 | 50° C. | 319.21 | 2.7 | 861.83 | 319.21 |
| 4:1 | pH 11 | 268.87 | 2.41 | 647.97 | 268.87 |
| 4:1 | pH 3 | 301.56 | 2.4 | 723.75 | 301.56 |
| 4:1 | Sonication | 334.93 | 2.12 | 710.06 | 334.93 |

*THC + THCA concentration were from Table 9 (μg/mL = μg/mg extract).
**Extract weight was from Table 8.

TABLE 13
Percentage total THC and CBD on cannabis sample based
on acetone/water (4:1) extracts under various conditions:
at 50° C., pH 3 and 11, and with aid of sonication.

| | | | Total CBD | CBD in | Total THC | THC in |
|---|---|---|---|---|---|---|
| Solvent | Extra | Sample | in Extract* | Sample | in Extract* | Sample |
| Ratio | Condition | (g) | (mg) | (%) | (mg) | (%) |
| 4:1 | 50° C. | 7.00 | 686.32 | 9.80 | 796.69 | 11.38 |
| 4:1 | pH 11 | 7.00 | 503.38 | 7.19 | 594.21 | 8.490 |
| 4:1 | pH 3 | 7.01 | 564.42 | 8.05 | 663.76 | 9.470 |
| 4:1 | Sonication | 6.05 | 561.21 | 9.28 | 651.39 | 10.77 |

| | | |
|---|---|---|
| Tweed - Percentage (%) CBD and THC in label | 7.00 | 10.20 |

*Cannabis sample weight/extract weight were from 8 and cannabinoids (THC, THCA, CBD & CBDA) concentrations were from 9. Total CBD and total THC concentration were calculated using following formula Total THC = THC + (THCA × 0.877) and Total CBD = CBD + (CBDA × 0.877)

### Example 3—Effects of Liquor to Solid Ratio and Retention Time on Extraction of Cannabinoids from Cannabis or Cannabis Waste

The efficiency of acetone/water mixture for extraction of cannabinoids was examined through varying acetone/water ratios and liquor/solid ratios against ethanol as a reference. The liquor to solid (L:S) ratio and retention time for cannabinoid extraction were also determined. Multiple batches (3.5 g) of commercial cannabis were obtained from Nova Scotia Liquor Corporation (Skunk Haze (Hybrid)), whole flower cannabis, containing 15.0 mg/g of THC (total THC 80 mg/g or 8%) and 6.53 mg/g CBD (total CBD 70.0 mg/g or 7.0%; Tweed Inc., Smiths Falls). The multiple batches of whole flower cannabis were combined and ground into a powder using a grinder. The powdered whole flower cannabis (1-10 g or 1 g scale) was extracted with acetone/water at various ratios and conditions. The initial experiments used acetone/water ratios of 4:1 and 5:1 (v/v) with liquor/solid ratios of 10:1. The timed experiments used acetone/water ratios of 4:1 with liquor/solid ratios of 8:1 and 6:1. The samples were prepared at room temperature and analyzed by LC/MS, calibration curves for the cannabinoid standards were generated, and cannabinoids concentrations were calculated as described in Example 1.

The results below suggest that the highest chemical extraction yields were obtained using a 4:1 acetone:water ratio, while the 5:1 acetone:water ratio had the highest cannabinoid yield. The optimum liquor/solid ratio was 10:1 with an overnight retention time (approximately 16 hours). The best extraction conditions appeared to be a 4:1 acetone:water ratio, 10:1 liquor/solid ratio, and 3 to 6 hours retention time.

TABLE 14
Percentage yield of acetone/water and EtOH extracts
at various conditions using 2-10 g scale extraction.
All extractions had an overnight retention time.

| Solvent | Sample wt | Extract wt | Yield | Ave Yield | |
|---|---|---|---|---|---|
| Ratio | (g) | (g) | (%) | (%) | SD |
| 4:1 | 2.04* | 0.58 | 28.43 | 29.32 | 2.42 |
| | 3.02* | 0.83 | 27.48 | | |
| | 10.01 | 3.21 | 32.06 | | |
| 5:1 | 10.01 | 2.43 | 24.28 | 26.09 | 1.62 |
| | 10.03 | 2.67 | 26.62 | | |
| | 10.01 | 2.74 | 27.37 | | |
| EtOH | 4.00* | 0.78 | 19.50 | 20.09 | 0.59 |
| | 4.02* | 0.80 | 19.90 | | |
| | 4.40* | 0.92 | 20.91 | | |
| | 10.03 | 2.01 | 20.04 | | |

TABLE 15
Summary of major cannabinoid concentration in μg/mg extract
determined by LC/MS (average of duplicate injections)

| Solvent | | | | | | | | | | | Total |
|---|---|---|---|---|---|---|---|---|---|---|---|
| Ratio | CBD | THC | CBDA | THCA | CBG | CBN | THCVA | THCV | CBGA | CBC | Cannabinoids |
| 4:1 | 26.8 | 45.7 | 171.3 | 151.7 | 2.5 | 3.9 | 1.1 | 0.3 | 6.8 | 1.7 | 411.8 |
| 5:1 | 26.3 | 42.7 | 230.8 | 243 | 2.6 | 4.1 | NA | — | 7.8 | 1.5 | 558.75 |
| | 31.1 | 48.5 | 248 | 252.5 | 2.9 | 4.5 | NA | — | 8.7 | 1.8 | 598.12 |
| | 26.2 | 42.9 | 221.1 | 227.8 | 2.5 | 3.9 | NA | — | 7.8 | 1.5 | 533.82 |
| EtOH | 35.6 | 57.3 | 266.2 | 281.1 | 3.2 | 4.9 | NA | — | 9.6 | 2.0 | 659.95 |

NA: Not analyzed
— : Not detected

TABLE 16
Total cannabinoids concentration in dry sample based
on the extract efficiency of acetone/water and EtOH

| | | | Total | | | Average | |
|---|---|---|---|---|---|---|---|
| | Total | Extract | Cannabinoids | Dry | Cannabinoids | Cannabinoids | |
| Solvent | Cannabinoids | wt. | in Extract | Sample | in Sample | in Sample | |
| Ratio | (mg/mg)* | (g)** | (mg) | (g) | (mg/g) | (mg/g) | SD |
| 4:1 | 411.8 | 3.21 | 1321.76 | 10.01 | 132.04 | 132.04 | — |
| 5:1 | 558.7 | 2.43 | 1357.8 | 10.01 | 135.64 | 146.99 | 11.82 |
| | 598.1 | 2.67 | 1597.0 | 10.03 | 159.22 | | |
| | 533.8 | 2.74 | 1462.7 | 10.01 | 146.12 | | |
| EtOH | 659.9 | 2.01 | 1326.49 | 10.03 | 132.25 | 132.25 | — |

*Total cannabinoid concentrations were from Table 15 (μg/mg extract)
**Extract weights and dry sample weights were obtained from Table 14

TABLE 17
Total CBD + CBDA concentration in acetone/water and EtOH extracts

| | | | Total | | Average | |
|---|---|---|---|---|---|---|
| | | Extract | CBD + CBDA | CBD + CBDA | CBD + CBDA | |
| Solvent | CBD + CBDA | wt | in Extract | in Extract | in Extract | |
| Ratio | (μg/mg)* | (g)** | (mg) | (mg/g) | (mg/g) | SD |
| 4:1 | 198.15 | 3.21 | 636.06 | 198.2 | 198.15 | — |
| 5:1 | 257.13 | 2.43 | 624.82 | 257.1 | 261.18 | 16.2 |
| | 279.05 | 2.67 | 745.06 | 279.1 | | |
| | 247.37 | 2.74 | 677.81 | 247.4 | | |
| EtOH | 301.78 | 2.01 | 606.57 | 301.8 | 301.78 | — |

*CBD and CBDA concentrations were obtained from Table 15 (μg/mL = μg/mg extract)
**Extract weights were obtained from Table 14

TABLE 18
Total THC + THCA concentrations in acetone/water and EtOH extracts

| | | Extract | Total THC | THC in | Average THC | |
|---|---|---|---|---|---|---|
| Solvent | THC + THCA | wt | in Extract | Extract | in Extract | |
| Ratio | (μg/mg)* | (g)** | (mg) | (mg/g) | (mg/g) | SD |
| 4:1 | 197.40 | 3.21 | 633.64 | 197.40 | 197.40 | — |
| 5:1 | 285.72 | 2.43 | 694.30 | 285.72 | 285.84 | 15.17 |
| | 301.07 | 2.67 | 803.85 | 301.07 | | |
| | 270.72 | 2.74 | 741.78 | 270.72 | | |
| EtOH | 338.46 | 2.01 | 680.30 | 338.46 | 338.46 | — |

*THC and THCA concentrations were obtained from Table 15 (μg/mL = μg/mg extract)
**Extract weights were obtained from Table 14

TABLE 19
Percentage of total CBD and total THC on new cannabis sample based on acetone/water
ratios (4:1 and 5:1) with various liquor/solid ratios and EtOH extracts.

| | | | | Total | | Average | Total | | Average |
|---|---|---|---|---|---|---|---|---|---|
| | Liquor/ | Retent. | | CBD in | CBD in | CBD in | THC in | THC in | THC in |
| Solvent | Solid | Time | Sample | Extract* | Sample | Sample | Extract* | Sample | Sample |
| Ratio | Ratio | (hour) | (g) | (mg) | (%) | (%) | (mg) | (%) | (%) |
| 4:1 | 10:1 | Over- | 10.01 | 568.42 | 5.68 | 5.68 | 573.76 | 5.73 | 5.73 |
| | | night | | | | | | | |
| 5:1 | 10:1 | Over- | 10.01 | 555.84 | 5.55 | 6.07 ± | 621.67 | 6.21 | 6.68 ± |
| | | night | 10.03 | 663.63 | 6.62 | 0.58 | 720.92 | 7.19 | 0.49 |
| | | | 10.01 | 603.28 | 6.03 | | 665.00 | 6.64 | |
| EtOH | 10:1 | Over- | 10.03 | 540.77 | 5.68 | 5.68 | 610.80 | 6.09 | 6.09 |
| | | night | | | | | | | |

| | | |
|---|---|---|
| Tweed - SKUNK HAZE Percentage (%) | 7.00 | 8.00 |
| of total CBD and total THC in product label | | |

Cannabis sample weight and extract weight were from Table 14. Total CBD and total THC concentration were calculated using following formula and the cannabinoids concentrations are shown in Table 15. Total THC = THC + (THCA × 0.877) and Total CBD = CBD + (CBDA × 0.877)

TABLE 20
Percentage yield of acetone/water ratio of
4:1 extracts at various liquor/solid ratios

| Liquor/ | Retention | Sample | Extract | | Ave. | |
|---|---|---|---|---|---|---|
| Solid | Time | wt | wt | Yield | Yield | |
| Ratio | (hour) | (g) | (mg) | (%) | (%) | SD |
| 6:1 | 1 | 1.04 | 256.3 | 24.64 | 25.38 | 1.04 |
| | | 1.01 | 263.8 | 26.12 | | |
| 6:1 | 3 | 1.03 | 245.5 | 23.83 | 23.15 | 0.97 |
| | | 1.04 | 233.6 | 22.46 | | |
| 6:1 | 6 | 1.04 | 254 | 24.42 | 24.81 | 0.55 |
| | | 1.03 | 259.5 | 25.19 | | |
| 6:1 | 8 | 1.01 | 259.6 | 25.70 | 25.30 | 0.57 |
| | | 1.02 | 254.0 | 24.90 | | |
| 8:1 | 1 | 1.00 | 250.2 | 25.02 | 24.76 | 0.37 |
| | | 1.04 | 254.7 | 24.49 | | |
| 8:1 | 3 | 1.04 | 270.2 | 25.98 | 25.63 | 0.49 |
| | | 1.02 | 257.9 | 25.28 | | |
| 8:1 | 6 | 1.00 | 269.2 | 26.92 | 27.50 | 0.83 |
| | | 1.01 | 283.7 | 28.09 | | |
| 8:1 | 8 | 1.02 | 272.4 | 26.71 | 26.26 | 0.64 |
| | | 1.03 | 265.8 | 25.81 | | |
| 10:1 | Overnight | 2.04 | 0.580 | 28.43 | 29.32 | 2.42 |
| | | 3.02 | 0.830 | 27.48 | | |
| | | 10.01 | 3.210 | 32.06 | | |

*Data were from Table 14.

TABLE 21
Summary of major cannabinoids concentration in μg/mg extract determined by LC/MS (average
of duplicate injections)*. All extractions were performed with an acetone/water ratio of 4:1.

| Liquor/ | Retent. | | | | | | | | | | | |
|---|---|---|---|---|---|---|---|---|---|---|---|---|
| Solid | Time | | | | | | | | | | | Total |
| Ratio | (hour) | CBD | THC | CBDA | THCA | CBG | CBN | THCVA | THCV | CBGA | CBC | Cannabinoids |
| 6:1 | 1 | 25.1 | 38.3 | 193.8 | 196.3 | 1.8 | 4.9 | 1.1 | 0.2 | 5.8 | 1.4 | 468.6 |
| | | 23.2 | 34.0 | 183.4 | 182.8 | 1.2 | 4.8 | 1.0 | 0.2 | 4.3 | 1.1 | 436.1 |
| | | 25.4 | 41.8 | 194.4 | 190.1 | 2.3 | 4.5 | 1.0 | 0.2 | 6.9 | 1.7 | 468.4 |
| 6:1 | 3 | 25.2 | 41.0 | 194.1 | 201.4 | 2.4 | 4.4 | 1.1 | 0.2 | 7.2 | 1.8 | 478.8 |
| | | 23.2 | 37.6 | 181.7 | 176.4 | 2.2 | 4.1 | 0.9 | 0.2 | 6.6 | 1.5 | 434.5 |
| 6:1 | 6 | 24.1 | 39.4 | 182.3 | 177.9 | 2.2 | 4.2 | 1.0 | 0.2 | 6.7 | 1.6 | 439.6 |
| | | 24.9 | 40.7 | 194.8 | 202.5 | 2.5 | 4.3 | 1.1 | 0.2 | 7.4 | 1.7 | 480.0 |
| 6:1 | 8 | 25.0 | 40.7 | 203.7 | 216.6 | 2.6 | 4.4 | 1.1 | 0.2 | 7.6 | 1.7 | 503.4 |
| | | 24.7 | 38.7 | 194.5 | 194.9 | 1.8 | 4.8 | 1.1 | 0.2 | 6.1 | 1.5 | 468.2 |
| 8:1 | 1 | 25.4 | 40.7 | 190.3 | 196.9 | 2.2 | 4.5 | 1.0 | 0.2 | 6.6 | 1.6 | 469.3 |
| | | 23.8 | 39.9 | 184.5 | 187.2 | 2.3 | 4.2 | 1.0 | 0.2 | 6.8 | 1.6 | 451.5 |
| 8:1 | 3 | 24.0 | 39.9 | 186.2 | 186.5 | 2.3 | 4.3 | 1.0 | 0.2 | 6.8 | 1.6 | 452.8 |
| | | 22.6 | 37.0 | 170.8 | 172.7 | 2.1 | 4.0 | 1.0 | 0.2 | 6.5 | 1.4 | 418.3 |
| 8:1 | 6 | 26.8 | 45.7 | 164.2 | 162.7 | 2.3 | 4.1 | 0.8 | 0.3 | 6.0 | 1.6 | 414.5 |
| 8:1 | 8 | 22.6 | 37.5 | 188.6 | 196.2 | 2.3 | 4.0 | 1.0 | 0.2 | 7.0 | 1.6 | 461.0 |
| | | 24.1 | 39.4 | 192.7 | 201.2 | 2.5 | 4.2 | 1.0 | 0.2 | 7.4 | 1.7 | 474.5 |
| 10:1 | Over- | 26.8 | 45.7 | 171.3 | 151.7 | 2.5 | 3.9 | 1.1 | 0.3 | 6.8 | 1.7 | 411.8 |
| | night | | | | | | | | | | | |

*Injection volume and sample preparation is described above.
*Data were from Table 15.

TABLE 22
Total cannabinoids concentration in dry sample based on the extract efficiency of
acetone/water. All extractions were performed with an acetone/water ratio of 4:1.

| | | | | Total | | | Average | |
|---|---|---|---|---|---|---|---|---|
| Liquor/ | Retent. | Total | Extract | Cannabinoids | Dry | Cannabinoids | Cannabinoids | |
| Solid | Time | Cannabinoids | wt | in Extract | Sample | in Sample | in Sample | |
| Ratio | (hour) | (mg/mg)* | (g)** | (mg) | (g) | (mg/g) | (mg/g) | SD |
| 6:1 | 1 | 468.6 | 0.26 | 120.10 | 1.04 | 115.48 | 114.69 | 1.12 |
| | | 436.1 | 0.26 | 115.03 | 1.01 | 113.89 | | |
| 6:1 | 3 | 468.4 | 0.25 | 114.99 | 1.03 | 111.64 | 109.60 | 2.89 |
| | | 478.8 | 0.23 | 111.85 | 1.04 | 107.55 | | |
| 6:1 | 6 | 434.5 | 0.25 | 110.35 | 1.04 | 106.11 | 108.43 | 3.28 |
| | | 439.6 | 0.26 | 114.07 | 1.03 | 110.75 | | |
| | 8 | 480.0 | 0.26 | 124.62 | 1.01 | 123.38 | 124.37 | 1.40 |
| | | 503.4 | 0.25 | 127.86 | 1.02 | 125.36 | | |
| 8:1 | 1 | 468.2 | 0.25 | 117.14 | 1.00 | 117.14 | 116.04 | 1.56 |
| | | 493.3 | 0.25 | 119.53 | 1.04 | 114.93 | | |
| 8:1 | 3 | 451.5 | 0.27 | 122.00 | 1.04 | 117.31 | 115.90 | 2.00 |
| | | 452.8 | 0.26 | 116.77 | 1.02 | 114.48 | | |
| 8:1 | 6 | 418.3 | 0.27 | 112.61 | 1.00 | 112.61 | 114.51 | 2.70 |
| | | 414.5 | 0.28 | 117.59 | 1.01 | 116.42 | | |
| 8:1 | 8 | 461.0 | 0.27 | 125.58 | 1.02 | 123.11 | 122.78 | 0.47 |
| | | 474.5 | 0.27 | 126.12 | 1.03 | 122.45 | | |
| 10:1* | Over- | 411.8 | 3.21 | 1321.76 | 10.01 | 132.04 | 132.04 | — |
| | night | | | | | | | |

*Total cannabinoid concentrations were from Tables 15 and 21 (mg/mg extract)
**Extract weights and dry sample weights were obtained from Tables 14 and 20.

TABLE 23
Total CBD + CBDA concentration in acetone/water extracts.
All extractions were performed with an acetone/water ratio of 4:1.

| | | | | Total | CBD + | Average | |
|---|---|---|---|---|---|---|---|
| Liquor/ | Retention | CBD + | Extract | CBD + | CBDA in | CBD + CBDA | |
| Solid | Time | CBDA | wt | CBDA in | Extract | in Extract | |
| Ratio | (hour) | (μg/mg)* | (g)** | Extract (mg) | (mg/g) | (mg/g) | SD |
| 6:1 | 1 | 218.88 | 0.2563 | 56.10 | 218.9 | 212.76 | 8.65 |
| | | 206.65 | 0.2638 | 54.51 | 206.7 | | |
| 6:1 | 3 | 219.86 | 0.2455 | 53.98 | 219.9 | 219.58 | 0.39 |
| | | 219.30 | 0.2336 | 51.23 | 219.3 | | |
| 6:1 | 6 | 204.93 | 0.2540 | 52.05 | 204.9 | 205.68 | 1.06 |
| | | 206.43 | 0.2595 | 53.57 | 206.4 | | |
| 6:1 | 8 | 219.67 | 0.2596 | 57.03 | 219.7 | 224.16 | 6.34 |
| | | 228.64 | 0.2540 | 58.07 | 228.6 | | |
| 8:1 | 1 | 219.16 | 0.2502 | 54.83 | 219.2 | 217.43 | 2.44 |
| | | 215.71 | 0.2547 | 54.94 | 215.7 | | |
| 8:1 | 3 | 208.28 | 0.2702 | 56.28 | 208.3 | 209.25 | 1.37 |
| | | 210.22 | 0.2579 | 54.21 | 210.2 | | |
| 8:1 | 6 | 193.40 | 0.2692 | 52.06 | 193.4 | 192.17 | 1.73 |
| | | 190.95 | 0.2837 | 54.17 | 191.0 | | |
| 8:1 | 8 | 211.13 | 0.2724 | 57.51 | 211.1 | 214.01 | 4.06 |
| | | 216.88 | 0.2658 | 57.65 | 216.9 | | |
| 10:1* | Overnight | 198.15 | 3.21 | 636.06 | 198.2 | 198.15 | — |

*CBD and CBDA concentrations were obtained from Tables 15 and 21 (μg/mL = μg/mg extract)
**Extract weights were obtained from Tables 14 and 20

TABLE 24
Total THC + THCA concentrations in acetone/water extracts.
All extractions were performed with an acetone/water ratio of 4:1.

| Liquor/ | Retention | THC + | Extract | Total THC | THC in | Average THC | |
|---|---|---|---|---|---|---|---|
| Solid | Time | THCA | Wt | in Extract | Extract | in Extract | |
| Ratio | (hour) | (μg/mg)* | (g)** | (mg) | (mg/g) | (mg/g) | SD |
| 6:1 | 1 | 234.58 | 0.256 | 60.12 | 234.58 | 225.66 | 12.6 |
| | | 216.75 | 0.264 | 57.18 | 216.75 | | |
| 6:1 | 3 | 231.93 | 0.246 | 50.94 | 231.93 | 237.18 | 7.43 |
| | | 242.44 | 0.234 | 56.63 | 242.44 | | |
| 6:1 | 6 | 214.01 | 0.254 | 54.36 | 214.01 | 215.63 | 2.29 |
| | | 217.25 | 0.260 | 56.38 | 217.25 | | |
| 6:1 | 8 | 243.22 | 0.260 | 63.14 | 243.22 | 250.23 | 9.92 |
| | | 257.25 | 0.254 | 65.34 | 257.25 | | |
| 8:1 | 1 | 233.59 | 0.250 | 58.44 | 233.59 | 235.55 | 2.78 |
| | | 237.52 | 0.255 | 60.50 | 237.52 | | |
| 8:1 | 3 | 227.11 | 0.270 | 61.36 | 227.11 | 226.78 | 0.46 |
| | | 226.45 | 0.258 | 58.40 | 226.45 | | |
| 8:1 | 6 | 209.75 | 0.269 | 56.46 | 209.75 | 209.11 | 0.90 |
| | | 208.48 | 0.284 | 58.14 | 208.48 | | |
| 8:1 | 8 | 233.69 | 0.272 | 63.66 | 233.69 | 237.16 | 4.92 |
| | | 240.64 | 0.266 | 63.96 | 240.54 | | |
| 10:1^{#} | Overnight | 197.4 | 3.21 | 633.64 | 197.4 | 197.4 | — |

*THC and THCA concentrations were obtained from Tables 15 and 21(μg/mL = μg/mg extract)
**Extract weights were obtained from Tables 14 and 20
^{#}Data were from Table 18.

TABLE 25
Percentage total THC and total CBD on new cannabis sample based on
acetone/water (4:1 and 5:1) with various liquor/solid ratios. All
extractions were performed with an acetone/water ratio of 4:1.

| | | | Total | | Average | Total | | Average |
|---|---|---|---|---|---|---|---|---|
| Liquor/ | Retent. | | CBD in | CBD in | CBD in | THC in | THC in | THC in |
| Solid | Time | Sample | Extract* | Sample | Sample | Extract* | Sample | Sample |
| Ratio | (hour) | (g) | (mg) | (%) | (%) | (mg) | (%) | (%) |
| 6:1 | 1 | 1.04 | 49.99 | 4.81 | 4.81 ± | 53.94 | 5.19 | 5.13 ± |
| | | 1.01 | 48.55 | 4.81 | 0.0002 | 51.26 | 5.08 | 0.08 |
| 6:1 | 3 | 1.03 | 48.09 | 4.67 | 4.53 ± | 51.19 | 4.97 | 4.93 ± |
| | | 1.04 | 45.65 | 4.39 | 0.20 | 50.84 | 4.89 | 0.06 |
| 6:1 | 6 | 1.04 | 46.37 | 4.46 | 4.55 ± | 48.84 | 4.70 | 4.81 ± |
| | | 1.03 | 47.74 | 4.64 | 0.13 | 50.71 | 4.92 | 0.16 |
| 6:1 | 8 | 1.01 | 50.81 | 5.03 | 5.05 ± | 56.67 | 5.61 | 5.68 ± |
| | | 1.02 | 51.73 | 5.07 | 0.03 | 58.59 | 5.74 | 0.09 |
| 8:1 | 1 | 1.00 | 48.86 | 4.89 | 4.80 ± | 52.45 | 5.24 | 5.24 ± |
| | | 1.04 | 48.98 | 4.71 | 0.13 | 54.35 | 5.23 | 0.01 |
| 8:1 | 3 | 1.04 | 50.15 | 4.82 | 4.78 ± | 55.14 | 5.30 | 5.22 ± |
| | | 1.02 | 48.30 | 4.74 | 0.06 | 52.47 | 5.14 | 0.11 |
| 8:1 | 6 | 1.00 | 46.41 | 4.64 | 4.72 ± | 50.73 | 5.07 | 5.18 ± |
| | | 1.01 | 48.46 | 4.80 | 0.11 | 53.45 | 5.28 | 0.15 |
| 8:1 | 8 | 1.02 | 51.21 | 5.02 | 5.00 ± | 57.09 | 5.60 | 5.58 ± |
| | | 1.03 | 51.33 | 4.98 | 0.03 | 57.37 | 5.57 | 0.02 |
| 10:1^{#} | Over- | 10.01 | 568.42 | 5.68 | 5.68 | 573.76 | 5.73 | 5.73 |
| | night | | | | | | | |

| | | |
|---|---|---|
| Tweed - SKUNK HAZE Percentage (%) of | 7.00 | 8.00 |
| total CBD and total THC in product label | | |

Cannabis sample weight and extract weight were from Tables 14 and 20. Total CBD and total THC concentration were calculated using following formula and the cannabinoids concentrations are shown in Tables 15 and 21.
^{#}Data were from Table 19.
Total TDC = THC + (THCA × 0.877) and Total CBD = CBD + (CBDA × 0.877)

### Example 4—Pilot-Scale Extraction of Cannabinoids from Cannabis

The method described in Example 1 was performed at pilot scale using larger amounts of hemp flower (11-13 kg wet or 1 kg dried material). The key performance indicators assessed included validation of extracted cannabinoids; determination of the extent of cannabinoids extraction; identification and quantification of the extracted cannabinoids, and the performance efficiency of pilot scale extraction.

To determine the chlorophyll content, sample vials were left uncovered in darkness (to prevent decomposition of CBD) in a fume hood for 48 hours to allow the acetone to evaporate. After 48 hours samples were volumized to 100 mL in ethanol. Samples were diluted until appropriate absorbance values were achieved (0.2-0.8 abs at A₆₆₃ and A₆₄₅) and read against an ethanol blank. Chlorophyll content was determined using a UV-VIS method (Amon, 1949, Plant Physiol. 24, 1-15) and calculated using the following formula (2):

$$Chlorophyll a ( \frac{mg}{mL} ) = 12.7 {A}_{662} - 2.69 {A}_{643} \qquad ( 2 )$$

$$Chlorophyll b ( \frac{mg}{mL} ) = 22.9 {A}_{645} - 4.68 {A}_{663}$$

$$Total Chlorophyll ( \frac{mg}{mL} ) = Chlorophyll a + Chlorophyll$$

where: A₆₄₅=absorbance at a wavelength of 645 nm and A₆₆₃=absorbance at a wavelength of 663 nm.

Sample information from six extracts (Table 26) was used to estimate the total amount of oil in each extract, ignoring solvent weight (Table 27). The solvent was acetone to water 4:1 (v/v). Experiments 4-6 were extracted at a 1:8 solid to solvent ratio. Experiments 7-9 were extracted as at 1:6 solid to solvent ratio.

TABLE 26
Sample information

| | Biomass | Total | Total | Total Distillate |
|---|---|---|---|---|
| | Used | Oil | Solvent | [Oil + Solvent] |
| Experiment | (g) | (g) | (g) | (g) |
| Experiment 4 (Lot 7) | 1132.01 | 131.84 | 189.12 | 320.96 |
| Experiment 5 (Lot 10) | 1199.16 | 170.20 | 147.55 | 317.75 |
| Experiment 6 (Lot 12) | 1065.08 | 131.91 | 207.35 | 338.96 |
| Experiment 7 (Lot 13) | 1035.03 | 105.58 | 168.41 | 274.09 |
| Experiment 8 (Lot 16) | 1092.92 | 311.16 | 163.42 | 474.58 |
| Experiment 9 (Lot 17) | 1323.68 | 154.03 | 446.13 | 600.16 |

TABLE 27
Approximate mass of oil in each sample

| | Total | Total | | Wt. | Approximate mass |
|---|---|---|---|---|---|
| | Oil | Solvent | Oil:Solvent | Sample | oil in sample |
| Experiment | (g) | (g) | Ratio | (g) | (g) |
| Experiment 4 (Lot 7) | 131.84 | 189.12 | 0.70 | 21.42 | 14.93 |
| Experiment 5 (Lot 10) | 170.20 | 147.55 | 1.15 | 20.99 | 24.21 |
| Experiment 6 (Lot 12) | 131.91 | 207.05 | 0.64 | 26.61 | 16.95 |
| Experiment 7 (Lot 13) | 105.68 | 168.41 | 0.63 | 32.79 | 20.58 |
| Experiment 8 (Lot 16) | 311.16 | 163.42 | 1.90 | 31.27 | 59.54 |
| Experiment 9 (Lot 17) | 154.03 | 446.13 | 0.35 | 28.58 | 9.87 |

TABLE 28
Sample preparation for chlorophyll analysis (serial dilution)

| | Approximate | | Concen- | Aliquot | Final |
|---|---|---|---|---|---|
| | mass oil | EtOH | tration | Volume | Volume |
| Experiment | (g) | (mL) | (g/mL) | (mL) | (mL) |
| Experiment 4 (Lot 7) | 14.93 | 100 | 0.1493 | 0.5 | 25 |
| Experiment 5 (Lot 10) | 24.21 | | 0.2421 | 0.5 | 25 |
| Experiment 6 (Lot 12) | 16.95 | | 0.1695 | 0.5 | 25 |
| Experiment 7 (Lot 13) | 20.58 | | 0.2058 | 0.5 | 25 |
| Experiment 8 (Lot 16) | 59.54 | | 0.5954 | 0.5 | 25 |
| Experiment 9 (Lot 17) | 9.87 | | 0.0987 | 0.5 | 25 |

TABLE 29
UV-VIS Abs for diluted samples and subsequent calculations
for chlorophyll a, chlorophyll b, and total chlorophyll

| | | | | | Total |
|---|---|---|---|---|---|
| | | | | | Chloro- |
| | | | Chloro- | Chloro- | phyll |
| | | | phyll a | phyll b | Conc. |
| Experiment | A₆₆₃ | A₆₄₅ | (mg/mL) | (mg/mL) | (mg/ml) |
| Experiment 4 (Lot 7) | 0.575 | 0.237 | 6.66497 | 2.7363 | 9.40127 |
| Experiment 5 (Lot 10) | 0.623 | 0.248 | 7.24498 | 2.76356 | 10.00854 |
| Experiment 6 (Lot 12) | 0.586 | 0.23 | 6.8235 | 2.52452 | 9.34802 |
| Experiment 7 (Lot 13) | 0.456 | 0.259 | 5.09449 | 3.79702 | 8.89151 |
| Experiment 8 (Lot 16) | 0.354 | 0.15 | 4.0923 | 1.77828 | 5.87058 |
| Experiment 9 (Lot 17) | 0.463 | 0.201 | 5.33941 | 2.43606 | 7.77547 |

TABLE 30
Total chlorophyll concentration in extracts
diluted to 100 mL with ethanol

| | Total Chlorophyll Concentration |
|---|---|
| Experiment | in 100 mL Oil + Ethanol (mg/mL) |
| Experiment 4 (Lot 7) | 470.0635 |
| Experiment 5 (Lot 10) | 500.427 |
| Experiment 6 (Lot 12) | 467.401 |
| Experiment 7 (Lot 13) | 444.5755 |
| Experiment 8 (Lot 16) | 293.529 |
| Experiment 9 (Lot 17) | 388.7735 |

Winterization (wax determination) was performed by dissolving the extracts into approximately 10 mL of ethanol per gram of estimated oil, then placed into an ultralow freezer (−80° C.) for 24 hours after which the extracts were vacuum filtered and collected before removal of the ethanol under reduced pressure. Winterization was performed twice per sample.

TABLE 31
Mass of solids and extract following the first winterization

| Experiment | Mass Solids (g) | Mass Extract (g) |
|---|---|---|
| Experiment 4 (Lot 7) | 3.849 | 10.177 |
| Experiment 5 (Lot 10) | 4.607 | 11.941 |
| Experiment 6 (Lot 12) | 5.741 | 11.818 |
| Experiment 7 (Lot 13) | 2.182 | 25.645 |
| Experiment 8 (Lot 16) | 5.078 | 8.430 |
| Experiment 9 (Lot 17) | 3.096 | 10.220 |

TABLE 32
Mass of solids and extract following the second winterization

| Experiment | Mass Solids (g) | Mass Extract (g) |
|---|---|---|
| Experiment 4 (Lot 7) | 0.689 | 9.888 |
| Experiment 5 (Lot 10) | 3.115 | 8.324 |
| Experiment 6 (Lot 12) | 0.633 | 19.665 |
| Experiment 7 (Lot 13) | 8.931 | 17.260 |
| Experiment 8 (Lot 16) | 0.177 | 8.689 |
| Experiment 9 (Lot 17) | 3.544 | 9.431 |

Following winterization, extracts were decolorized using activated charcoal. Extracts were dissolved in aqueous 80% ethanol (approximately 200 mL). Activated charcoal was added directly to the dissolved extracts, shaken vigorously for 1 minute, then centrifuged. Multiple attempts were performed before finding a mass of activated charcoal (7 grams/sample) that made a significant visual difference to the color of the finished extracts. More activated charcoal could have likely been used for further decolorization, though this was not risked due to the possibility of adsorbing CBD and reducing extract potency.

TABLE 33
Mass of finished extract after decolorization
with activated charcoal

| | Mass Activated | Mass Finished |
|---|---|---|
| Experiment | Charcoal | Extract (g) |
| Experiment 4 (Lot 7) | Approximately 7 g | 5.631 |
| Experiment 5 (Lot 10) | | 5.173 |
| Experiment 6 (Lot 12) | | 7.770 |
| Experiment 7 (Lot 13) | | 12.45 |
| Experiment 8 (Lot 16) | | 4.401 |
| Experiment 9 (Lot 17) | | 4.125 |

TABLE 34
CBDa, CBD, and total CBD concentrations
in processed extracts (triplicate)

| | CBDa | CBD | |
|---|---|---|---|
| | Concentration | % w/w | Total |
| | finished extract | finished | CBD |
| Experiment and Lot Number | (% w/w) | extract | (% w/w) |
| Experiment 4 (Lot 7) | 19.722 | 7.190 | 24.486 |
| | 9.053 | 3.260 | 11.199 |
| | 11.689 | 4.229 | 14.480 |
| Experiment 5 (Lot 10) | 27.146 | 9.975 | 33.782 |
| | 27.681 | 10.023 | 34.299 |
| | 28.457 | 10.216 | 35.173 |
| Experiment 6 (Lot 12) | 10.073 | 3.847 | 12.681 |
| | 7.706 | 2.948 | 9.706 |
| | 7.323 | 2.790 | 9.212 |
| Experiment 7 (Lot 13) | 3.240 | 1.109 | 3.950 |
| | 2.571 | 0.876 | 3.131 |
| | 3.059 | 1.048 | 3.731 |
| Experiment 8 (Lot 16) | 5.916 | 1.460 | 6.648 |
| | 8.380 | 2.070 | 9.419 |
| | 7.883 | 1.920 | 8.833 |
| Experiment 9 (Lot 17) | 19.665 | 7.147 | 24.393 |
| | 15.262 | 5.580 | 18.965 |
| | 20.989 | 7.679 | 26.086 |

TABLE 35
Average total CBD content of processed extracts and
calculated total CBD content of crude extracts

| | Average of | Weight of | |
|---|---|---|---|
| | Total CBD | Total CBD | Total CBD |
| | in Processed | in Cleaned | in Extract |
| | Extract | up extract | as received |
| Experiment and Lot Number | (% w/w) | (g) | (% w/w)* |
| Experiment 4 (Lot 7) | 16.722 | 0.942 | 10.702 |
| Experiment 5 (Lot 10) | 34.418 | 1.780 | 15.836 |
| Experiment 6 (Lot 12) | 10.533 | 0.818 | 7.903 |
| Experiment 7 (Lot 13) | 3.604 | 0.449 | 3.549 |
| Experiment 8 (Lot 16) | 8.300 | 0.365 | 1.782 |
| Experiment 9 (Lot 17) | 23.148 | 0.955 | 13.018 |

*Assuming No Loss of Cannabinoids through Cleanup Processes*

TABLE 36
Terpene potency of processed extracts

| Date: 26 Nov. 2021 | | Concentrations of Analyte in Sample (% w/w) | | | | | | | | | |
|---|---|---|---|---|---|---|---|---|---|---|---|
| Sample | Sample | α- | β- | β- | D- | | Caryophyllene | | | Mass of Sample | |
| Group | Name | pinene | myrcene | pinene | limonene | Linalool | Oxide | Humulene | Nerolidol | (mg) | (μg) |
| 7 | 7a | 0.000 | 0.000 | 0.000 | 0.000 | 0.000 | 0.575 | 0.000 | 0.000 | 1.260 | 1260 |
| | 7b | 0.000 | 0.000 | 0.000 | 0.000 | 0.000 | 0.819 | 0.000 | 0.000 | 1.116 | 1116 |
| | 7c | 0.000 | 0.000 | 0.000 | 0.000 | 0.000 | 0.856 | 0.000 | 0.000 | 1 | 1000 |
| 10 | 10a | 0.000 | 0.000 | 0.000 | 0.000 | 0.000 | 0.754 | 0.000 | 0.000 | 1.08 | 1080 |
| | 10b | 0.000 | 0.000 | 0.000 | 0.000 | 0.000 | 0.717 | 0.000 | 0.000 | 1.13 | 1130 |
| | 10c | 0.000 | 0.000 | 0.000 | 0.000 | 0.000 | 0.710 | 0.000 | 0.000 | 1.17 | 1170 |
| 12 | 12a | 0.000 | 0.000 | 0.000 | 0.000 | 0.000 | 0.361 | 0.000 | 0.000 | 1.28 | 1280 |
| | 12b | 0.000 | 0.000 | 0.000 | 0.000 | 0.000 | 0.348 | 0.000 | 0.000 | 1.28 | 1280 |
| | 12c | 0.000 | 0.000 | 0.000 | 0.000 | 0.000 | 0.581 | 0.000 | 0.000 | 1.17 | 1170 |
| 13 | 13a | 0.000 | 0.000 | 0.000 | 0.000 | 0.000 | 0.354 | 0.000 | 0.000 | 1.1 | 1100 |
| | 13b | 0.000 | 0.000 | 0.000 | 0.000 | 0.000 | 0.344 | 0.000 | 0.000 | 1.19 | 1190 |
| | 13c | 0.000 | 0.000 | 0.000 | 0.000 | 0.000 | 0.411 | 0.000 | 0.000 | 1.02 | 1020 |
| 16 | 16a | 0.000 | 0.000 | 0.000 | 0.000 | 0.000 | 0.652 | 0.000 | 0.000 | 1.05 | 1050 |
| | 16b | 0.000 | 0.000 | 0.000 | 0.000 | 0.000 | 0.612 | 0.000 | 0.000 | 1.06 | 1060 |
| | 16c | 0.000 | 0.000 | 0.000 | 0.000 | 0.000 | 0.555 | 0.000 | 0.000 | 1.15 | 1150 |
| 17 | 17a | 0.000 | 0.000 | 0.000 | 0.000 | 0.000 | 0.597 | 0.000 | 0.000 | 1.21 | 1210 |
| | 17b | 0.000 | 0.000 | 0.000 | 0.000 | 0.000 | 0.661 | 0.000 | 0.000 | 1.09 | 1090 |
| | 17c | 0.000 | 0.000 | 0.000 | 0.000 | 0.000 | 0.672 | 0.000 | 0.000 | 1.06 | 1060 |

TABLE 37
Calculated terpene potency of crude extracts

| Date: 26 Nov. 2021 | | Concentrations of Analyte in Sample (% w/w) | | | | | | | | | |
|---|---|---|---|---|---|---|---|---|---|---|---|
| Sample | Sample | α- | β- | β- | D- | | Caryophyllene | | | Mass of Sample | |
| Group | Name | pinene | myrcene | pinene | limonene | Linalool | Oxide | Humulene | Nerolidol | (mg) | (μg) |
| 7 | 7a | 0.000 | 0.000 | 0.000 | 0.000 | 0.000 | 0.368 | 0.000 | 0.000 | 1.260 | 1260 |
| | 7b | 0.000 | 0.000 | 0.000 | 0.000 | 0.000 | 0.524 | 0.000 | 0.000 | 1.116 | 1116 |
| | 7c | 0.000 | 0.000 | 0.000 | 0.000 | 0.000 | 0.548 | 0.000 | 0.000 | 1 | 1000 |
| 10 | 10a | 0.000 | 0.000 | 0.000 | 0.000 | 0.000 | 0.342 | 0.000 | 0.000 | 1.08 | 1080 |
| | 10b | 0.000 | 0.000 | 0.000 | 0.000 | 0.000 | 0.325 | 0.000 | 0.000 | 1.13 | 1130 |
| | 10c | 0.000 | 0.000 | 0.000 | 0.000 | 0.000 | 0.322 | 0.000 | 0.000 | 1.17 | 1170 |
| 12 | 12a | 0.000 | 0.000 | 0.000 | 0.000 | 0.000 | 0.270 | 0.000 | 0.000 | 1.28 | 1280 |
| | 12b | 0.000 | 0.000 | 0.000 | 0.000 | 0.000 | 0.261 | 0.000 | 0.000 | 1.28 | 1280 |
| | 12c | 0.000 | 0.000 | 0.000 | 0.000 | 0.000 | 0.436 | 0.000 | 0.000 | 1.17 | 1170 |
| 13 | 13a | 0.000 | 0.000 | 0.000 | 0.000 | 0.000 | 0.349 | 0.000 | 0.000 | 1.1 | 1100 |
| | 13b | 0.000 | 0.000 | 0.000 | 0.000 | 0.000 | 0.339 | 0.000 | 0.000 | 1.19 | 1190 |
| | 13c | 0.000 | 0.000 | 0.000 | 0.000 | 0.000 | 0.404 | 0.000 | 0.000 | 1.02 | 1020 |
| 16 | 16a | 0.000 | 0.000 | 0.000 | 0.000 | 0.000 | 0.140 | 0.000 | 0.000 | 1.05 | 1050 |
| | 16b | 0.000 | 0.000 | 0.000 | 0.000 | 0.000 | 0.131 | 0.000 | 0.000 | 1.06 | 1060 |
| | 16c | 0.000 | 0.000 | 0.000 | 0.000 | 0.000 | 0.119 | 0.000 | 0.000 | 1.15 | 1150 |
| 17 | 17a | 0.000 | 0.000 | 0.000 | 0.000 | 0.000 | 0.336 | 0.000 | 0.000 | 1.21 | 1210 |
| | 17b | 0.000 | 0.000 | 0.000 | 0.000 | 0.000 | 0.371 | 0.000 | 0.000 | 1.09 | 1090 |
| | 17c | 0.000 | 0.000 | 0.000 | 0.000 | 0.000 | 0.378 | 0.000 | 0.000 | 1.06 | 1060 |

### Example 5—Extraction of Cannabinoids from Cannabis Waste Trim/Sugar Leaves

The method described in Example 1 can be used to extract cannabinoids from cannabis waste in the form of trim or sugar leaves, with results shown below.

TABLE 38
Percentage extraction yield of Acetone/water and EtOH extracts

| | Solvent | Sample | Extract | Yield | Ave Yield | |
|---|---|---|---|---|---|---|
| Sample | Acetone/water | wt (g) | wt (g) | (%) | (%) | SD |
| Cannabis | 4:1 | 10.05 | 3.29 | 32.74 | 32.54 | 0.78 |
| | 4:1 | 10.04 | 3.18 | 31.67 | | |
| | 4:1 | 10.03 | 3.33 | 33.20 | | |
| | 5:1 | 10.03 | 3.32 | 33.10 | 32.83 | 0.46 |
| | 5:1 | 10.00 | 3.31 | 33.10 | | |
| | 5:1 | 10.03 | 3.24 | 32.30 | | |
| | EtOH | 10.03 | 2.71 | 27.02 | 27.20 | 1.36 |
| | EtOH | 10.02 | 2.87 | 28.64 | | |
| | EtOH | 10.02 | 2.6 | 25.95 | | |
| Hemp | 4:1 | 10.04 | 1.79 | 17.83 | 18.26 | 0.39 |
| | 4:1 | 10.00 | 1.86 | 18.60 | | |
| | 4:1 | 10.03 | 1.84 | 18.34 | | |
| | 5:1 | 10.04 | 1.61 | 16.04 | 16.50 | 0.43 |
| | 5:1 | 10.00 | 1.69 | 16.90 | | |
| | 5:1 | 10.03 | 1.66 | 16.55 | | |
| | EtOH | 10.00 | 1.44 | 14.40 | 14.04 | 0.32 |
| | EtOH | 10.04 | 1.4 | 13.94 | | |
| | EtOH | 10.02 | 1.38 | 13.77 | | |

TABLE 39
Summary of major cannabinoid concentration in μg/mg extract determined by LC/MS (average of duplicate injections)

| | | | | | | | | | | | | | Total |
|---|---|---|---|---|---|---|---|---|---|---|---|---|---|
| Sample | Solvent | CBD | THC | CBDA | THCA | CBG | CBN | THCVA | THCV | CBGA | CBC | CBDV | Cannabinoids |
| Cannabis | 4:1 | — | 44.4 | 1.1 | 588.0 | 1.0 | — | 1.8 | — | 5.0 | 0.4 | — | 641.6 |
| | 4:1 | — | 26.1 | 0.8 | 434.5 | 0.4 | — | 1.7 | — | 3.8 | 0.2 | — | 467.4 |
| | 4:1 | — | 58.7 | 1.0 | 529.1 | 0.9 | — | 1.7 | — | 4.5 | 0.4 | — | 596.5 |
| | 5:1 | — | 44.3 | 0.9 | 458.0 | 0.6 | — | 1.6 | — | 4.1 | 0.4 | — | 510.0 |
| | 5:1 | — | 27.5 | 0.9 | 464.0 | 0.4 | — | 1.8 | — | 4.0 | 0.3 | — | 499.0 |
| | 5:1 | — | 48.6 | 1.0 | 519.1 | 0.8 | — | 1.8 | — | 4.5 | 0.4 | — | 576.1 |
| | EtOH | — | 26.7 | 1.1 | 607.2 | 0.7 | — | 2.2 | — | 4.9 | 0.3 | — | 643.1 |
| | EtOH | — | 30.4 | 1.0 | 547.4 | 0.6 | — | 2.0 | — | 4.6 | 0.3 | — | 586.4 |
| | EtOH | — | 29.9 | 1.0 | 558.3 | 0.7 | — | 2.1 | — | 4.9 | 0.3 | — | 597.2 |
| Hemp | 4:1 | 22.0 | 0.6 | 105.9 | 2.3 | — | — | — | — | 0.8 | 1.9 | 1.2 | 133.6 |
| | 4:1 | 25.7 | 0.7 | 124.5 | 1.7 | — | — | — | — | 1.0 | 2.1 | 1.4 | 155.7 |
| | 4:1 | 13.9 | 0.2 | 66.5 | 1.2 | — | — | — | — | 0.7 | 1.2 | 0.7 | 83.7 |
| | 5:1 | 28.5 | 0.8 | 145.2 | 2.0 | — | — | — | — | 1.2 | 2.5 | 1.6 | 180.3 |
| | 5:1 | 20.5 | 0.5 | 96.1 | 1.6 | — | — | — | — | 0.8 | 1.9 | 1.1 | 121.5 |
| | 5:1 | 33.6 | 1.0 | 161.0 | 2.7 | — | — | — | — | 1.3 | 2.7 | 1.8 | 202.4 |
| | EtOH | 33.9 | 1.0 | 167.3 | 2.6 | — | — | — | — | 1.4 | 3.0 | 1.8 | 209.1 |
| | EtOH | 42.9 | 1.5 | 216.3 | 3.7 | — | — | — | — | 1.6 | 4.0 | 2.4 | 269.9 |
| | EtOH | 38.5 | 1.2 | 196.4 | 3.3 | — | — | — | — | 1.5 | 3.4 | 2.1 | 244.4 |

TABLE 40
Total cannabinoid concentration in dry sample based on the
extract efficiency of acetone/water and EtOH extract

| | | | | Total | | Ave | |
|---|---|---|---|---|---|---|---|
| | | Sample | Ext | Cannabinoids | Cannabinoids | cannabinoids | |
| | | wt | weight | in extract or in | in sample | in sample | |
| Sample | Solvent | (g)* | (g)* | sample (μg)** | (mg/g) | (mg/g) | SD |
| Cannabis | 4:1 | 10.05 | 3.29 | 2110801 | 210.03 | 185.37 | 32.87 |
| | 4:1 | 10.04 | 3.18 | 1486363 | 148.04 | | |
| | 4:1 | 10.03 | 3.33 | 1986205 | 198.03 | | |
| | 5:1 | 10.03 | 3.32 | 1693067 | 168.80 | 173.35 | 11.19 |
| | 5:1 | 10.00 | 3.31 | 1651531 | 165.15 | | |
| | 5:1 | 10.03 | 3.24 | 1866534 | 186.10 | | |
| | EtOH | 10.03 | 2.71 | 1742694 | 173.75 | 165.56 | 9.61 |
| | EtOH | 10.02 | 2.87 | 1682933 | 167.96 | | |
| | EtOH | 10.02 | 2.60 | 1552822 | 154.97 | | |
| Hemp | 4:1 | 10.04 | 1.79 | 239068 | 23.81 | 22.71 | 6.88 |
| | 4:1 | 10.00 | 1.86 | 289691 | 28.97 | | |
| | 4:1 | 10.03 | 1.84 | 153943 | 15.35 | | |
| | 5:1 | 10.04 | 1.61 | 290358 | 28.92 | 27.65 | 6.58 |
| | 5:1 | 10.00 | 1.69 | 205341 | 20.53 | | |
| | 5:1 | 10.03 | 1.66 | 336017 | 33.50 | | |
| | EtOH | 10.00 | 1.44 | 301156 | 30.12 | 33.80 | 3.76 |
| | EtOH | 10.04 | 1.40 | 377892 | 37.64 | | |
| | EtOH | 10.02 | 1.38 | 337266 | 33.66 | | |

*Extract weight and dry sample weight were from Table 38.
**Total cannabinoids concentration was from Table 39 (μg/mg extract).

### Example 6—Extraction of Echinacea, Rhodiola, and Lion's Mane Mushrooms

Experiments were conducted to extract phytochemicals from Echinacea, Rhodiola, and Lion's Mane mushrooms using an extraction solution of acetone and water prepared at different ratios and performing extraction at room temperature. Echinacea, Rhodiola, or Lion's Mane powder (approximately 10 g) was extracted in triplicates. Plant/fungal material was added to a 250 mL round bottom flask charged with a stir bar, and solvent was added directly to this flask. The mixture was stirred overnight at room temperature. The next morning (16 hours stirring) extracts were vacuum filtered using a Buchner funnel, wetting the filter will approximately 2 mL ultrapure water. The round bottom flasks were rinsed with approximately 5 mL ultrapure water (2×) to transfer any remaining solids. The filtrate was dried under reduced pressure using a rotary evaporator, and the temperature did not exceed 40° C. during the evaporation process. Ethanolic extracts were evaporated to dryness. After partial evaporation, the concentrated filtrate of the acetonic extracts (water; all acetone should be evaporated) was transferred to a pre-weighed freeze-drier vessel. The flask was rinsed with 5 mL ultrapure water (2×) to transfer any remaining residue to the freeze-drier vessel. The samples were freeze-dried, weighed, and stored in the freezer for future use.

Echinacea and Rhodiola extracts were dissolved in approximately 20 mL (in the case of ethanolic extracts) or 40 mL (in the case of acetonic extracts) of methanol. Ethanolic extracts near-completely dissolved and were volumized to 25 mL prior to being centrifuged and placed into vials for HPLC analysis. Acetonic extracts had significant amounts of insoluble material (water-soluble after investigation) remaining after the addition of methanol. These acetonic extracts in methanol were sonicated at 40° C. for 10 mins, prior to being decanted and volumized to 50 mL. After volumizing, the extracts were centrifuged and placed into vials for HPLC analysis. The crude yields from each set of extractions are detailed in the Tables below.

The quantities of Echinacea biomarkers (caftaric acid, echinacoside, and chicoric acid) were analyzed by HPLC. External standards (caftaric acid and echinacoside from Sigma Aldrich; chicoric acid from Toronto Research Chemicals) were used in the quantification and were prepared individually (as opposed to a mixed standard), and independent calibration curves were created (data not shown). Throughout the runs, retention time shifting was observed, creating difficulties with the qualification of the biomarkers. This was especially troublesome with caftaric acid and chicoric acid, which exhibited much lower response in the extracts than echinacoside and were detected in retention time regions occupied by unknown minor peaks. To validate the assignment of each peak, samples which exhibited high retention time drifts were spiked with a 3-component addition standard containing caftaric acid, echinacoside, and chicoric acid. Following initial observations, appropriate calibration levels were constructed to ensure that samples fell within the calibration window.

TABLE 41
Crude extraction yields for Echinacea

| | | | | | Avg. | |
|---|---|---|---|---|---|---|
| | | Sample | Extract | Yield | Yield | % |
| Solvent Ratio | Extraction | (g) | (g) | (%) | (%) | RSD |
| 1:0 Ethanol | E1 | 9.907 | 0.438 | 4.42 | 3.88 | 26.42 |
| | E2 | 9.957 | 0.450 | 4.52 | | |
| | E3 | 10.084 | 0.272 | 2.70 | | |
| 4:1 Acetone:water | E4 | 10.185 | 2.102 | 20.64 | 20.47 | 17.95 |
| | E5 | 10.361 | 1.732 | 16.72 | | |
| | E6 | 10.242 | 2.464 | 24.06 | | |
| 5:1 Acetone:water | E7 | 10.395 | 2.330 | 22.41 | 18.82 | 16.54 |
| | E8 | 10.204 | 1.738 | 17.03 | | |
| | E9 | 10.010 | 1.703 | 17.01 | | |

TABLE 42
Calibration levels used in the final quantification
of Echinacea biomarkers in Echinacea extracts

| Biomarker | Calibration levels | |
|---|---|---|
| Caftaric acid | 5, 25, 50 | ppm |
| Echinacoside | 125, 250, 500 | ppm |
| Chicoric acid | 10, 30, 50 | ppm |

TABLE 43
Quantification of biomarker compounds
in Echinacea extracts (mg/extract)

| Sample | Chicoric acid | Echinacoside | Caftaric acid |
|---|---|---|---|
| Name | (mg) | (mg) | (mg) |
| E1 | ND | 0.454 | 0.006 |
| E2 | ND | 0.774 | 0.010 |
| E3 | ND | 0.386 | 0.005 |
| E4 | 0.026 | 4.012 | 0.048 |
| E5 | 0.020 | 3.172 | 0.038 |
| E6 | 0.023 | 3.478 | 0.044 |
| E7 | 0.035 | 5.708 | 0.066 |
| E8 | 0.016 | 2.929 | 0.039 |
| E9 | 0.026 | 4.332 | 0.052 |

TABLE 44
Average quantity of Echinacea biomarker compounds
in each series of extractions (mg/extract)

| Sample | Chicoric acid | Echinacoside | Caftaric acid |
|---|---|---|---|
| Name | (mg) | (mg) | (mg) |
| 1:0 | ND | 0.538 | 0.007 |
| Ethanol | | | |
| 4:1 | 0.023 | 3.554 | 0.043 |
| acetone:water | | | |
| 5:1 | 0.027 | 4.322 | 0.052 |
| acetone:water | | | |

The quantities of Rhodiola biomarkers (salidroside, rosarin, and rosavin) were analyzed by HPLC. External standards (salidroside, rosarin, rosavin from Sigma Aldrich) were used in the quantification, and were prepared individually (as opposed to a mixed standard), and independent calibration curves were created (data not shown). Significant baseline drift was observed during the runs at a detection wavelength of 205 nm. The baseline drift was appreciably reduced by changing the detection wavelength to 254 nm; however, salidroside is not detectable at this wavelength. Salidroside was thus quantified using detection at 205 nm, while rosarin and rosavin were quantified using detection at 254 nm.

TABLE 45
Crude extraction yields of Rhodiola

| | | | | | Avg. | |
|---|---|---|---|---|---|---|
| | | Sample | Extract | Yield | Yield | % |
| Solvent ratio | Extraction | (g) | (g) | (%) | (%) | RSD |
| 1:0 Ethanol | R1 | 10.163 | 0.606 | 5.96 | 3.81 | 49.66 |
| | R2 | 10.123 | 0.243 | 2.40 | | |
| | R3 | 9.993 | 0.307 | 3.07 | | |
| 4:1 Acetone:water | R4 | 10.021 | 3.441 | 34.34 | 30.25 | 11.72 |
| | R5 | 10.273 | 2.871 | 27.95 | | |
| | R6 | 10.397 | 2.961 | 28.48 | | |
| 5:1 Acetone:water | R7 | 10.241 | 2.592 | 25.31 | 26.34 | 6.66 |
| | R8 | 10.613 | 3.010 | 28.36 | | |
| | R9 | 10.320 | 2.615 | 25.34 | | |

TABLE 46
Quantification of biomarker compounds
in Rhodiola extracts (mg/extract)

| Sample | Salidraside | Rosarin | Rosavin |
|---|---|---|---|
| Name | (mg) | (mg) | (mg) |
| R1 | 0.175 | 0.091 | 0.251 |
| R2 | 0.053 | 0.028 | 0.080 |
| R3 | 0.083 | 0.044 | 0.122 |
| R4 | 4.511 | 2.646 | 6.372 |
| R5 | 3.095 | 1.804 | 4.476 |
| R6 | 3.500 | 2.025 | 5.110 |
| R7 | 3.612 | 2.125 | 5.440 |
| R8 | 3.741 | 2.115 | 5.369 |
| R9 | 3.567 | 2.013 | 5.108 |

TABLE 47
Average quantity of Rhodiola biomarker compounds
in each series of extractions (mg/extract)

| Sample | Salidroside | Rosarin | Rosavin |
|---|---|---|---|
| Name | (mg) | (mg) | (mg) |
| 1:0 | 0.104 | 0.054 | 0.151 |
| Ethanal | | | |
| 4:1 | 3.702 | 2.158 | 5.320 |
| acetone:water | | | |
| 5:1 | 3.640 | 2.084 | 5.306 |
| acetone:water | | | |

The beta-glucans in each Lion's Mane mushroom extract were quantified using a Megazyme β-Glucan (Yeast & Mushroom) Assay Kit (Megazyme Ltd.) suitable for measurement and analysis of 1,3:1,6-β-glucan and α-glucan in mushroom preparations.

TABLE 48
Crude extraction yields of Lion's Mane mushrooms

| | | | | | Avg. | |
|---|---|---|---|---|---|---|
| | | Sample | Extract | Yield | Yield | % |
| Solvent ratio | Extraction | (g) | (g) | (%) | (%) | RSD |
| 1:0 Ethanol | M1 | 10.055 | 2.761 | 27.46 | 21.10 | 31.55 |
| | M2 | 10.036 | 2.104 | 20.96 | | |
| | M3 | 10.067 | 1.499 | 14.89 | | |
| 4:1 Acetone:water | M4 | 10.01 | 1.641 | 16.39 | 16.30 | 2.58 |
| | M5 | 10.07 | 1.597 | 15.86 | | |
| | M6 | 10.015 | 1.668 | 16.66 | | |
| 5:1 Acetone:water | M7 | 10.08 | 1.41 | 13.99 | 13.90 | 1.85 |
| | M8 | 10.017 | 1.412 | 14.10 | | |
| | M9 | 10.031 | 1.365 | 13.61 | | |

TABLE 49
Beta-glucan content of Lion's Mane mushroom extracts

| Sample | | | Average |
|---|---|---|---|
| Name | Analyte | (% w/w) | Beta-Glucan |
| M1 | Total Glucan | 1.696 | 1.759 |
| | Alpha-Glucan | 0.264 | |
| | Beta-Glucan | 1.432 | |
| M2 | Total Glucan | 2.223 | |
| | Alpha-Glucan | 0.218 | |
| | Beta-Glucan | 2.005 | |
| M3 | Total Glucan | 2.120 | |
| | Alpha-Glucan | 0.279 | |
| | Beta-Glucan | 1.841 | |
| M4 | Total Glucan | 1.398 | 1.628 |
| | Alpha-Glucan | 0.279 | |
| | Beta-Glucan | 1.120 | |
| M5 | Total Glucan | 2.078 | |
| | Alpha-Glucan | 0.222 | |
| | Beta-Glucan | 1.856 | |
| M6 | Total Glucan | 2.075 | |
| | Alpha-Glucan | 0.167 | |
| | Beta-Glucan | 1.907 | |
| M7 | Total Glucan | 1.997 | 1.901 |
| | Alpha-Glucan | 0.540 | |
| | Beta-Glucan | 1.458 | |
| M8 | Total Glucan | 2.307 | |
| | Alpha-Glucan | 0.311 | |
| | Beta-Glucan | 1.996 | |
| M9 | Total Glucan | 2.588 | |
| | Alpha-Glucan | 0.341 | |
| | Beta-Glucan | 2.248 | |

From the above experiments, the 4:1 acetone:water solvent system resulted in the highest crude yields for Echinacea and Rhodiola extractions. Both 4:1 and 5:1 acetone:water solvent systems produced extracts with greater quantities of biomarker compounds compared to ethanol produced extracts for Echinacea and Rhodiola extractions. The ethanol solvent system resulted in the highest crude yield for Lion's Mane mushroom extractions, followed by the 4:1 acetone:water solvent system. Ethanol, 4:1 acetone:water, and 5:1 acetone:water solvent systems produced Lion's Mane mushroom extracts with similar quantities of beta-glucans.

As will be apparent to those skilled in the art, various modifications, adaptations and variations of the foregoing specific disclosure can be made without departing from the scope of the invention claimed herein.

What is claimed is:

1. A method of preparing an extract of a plant, fungus, waste, or part thereof comprising:
contacting a powder obtained from the plant, fungus, waste, or part thereof with an extraction solution comprising acetone and water at a predetermined ratio, followed by filtration and solvent removal to obtain the extract comprising one or more phytocannabinoids, cannabimimetic compounds, terpenes, flavonoids, alkaloids, waxes, and other secondary metabolites.

2. The method of claim 1, wherein the predetermined ratio of acetone to water in the extraction solution ranges from 1:0 (v/v) to 5:1 (v/v).

3. The method of claim 2, wherein the predetermined ratio of acetone to water in the extraction solution is selected from 1:0 (v/v), 1:2 (v/v), 1:1 (v/v), 3:2 (v/v), 2:1 (v/v), 4:1 (v/v), or 5:1 (v/v).

4. The method of claim 3, wherein the predetermined ratio of acetone to water in the extraction solution is selected from 4:1 (v/v) or 5:1 (v/v).

5. The method of claim 2, wherein extraction is conducted under one or more conditions of:
(i) a time ranging from at least about 30 minutes to about 16 hours;
(ii) a temperature ranging from about −40° C. to about 60° C.;
(iii) a pH ranging from 3.0 to 11.0;
(iv) a liquor to solid ratio ranging from 6:1 to 10:1; and
(v) sonication.

6. The method of claim 5, wherein extraction is conducted for at least about 30 minutes to about 6 hours.

7. The method of claim 5, wherein extraction is conducted at about room temperature.

8. The method of claim 5, wherein extraction is conducted at about 50° C.

9. The method of claim 8, wherein the predetermined ratio of acetone to water in the extraction solution is 4:1 (v/v), and extraction is conducted at about 50° C. with sonication.

10. The method of claim 5, wherein the predetermined ratio of acetone to water in the extraction solution is 4:1 (v/v), and extraction is conducted at a liquor to solid ratio of 10:1 for about 3 hours to about 6 hours.

11. The method of claim 1, wherein the one or more phytocannabinoids comprise delta-9-THC, delta-8-THC, iso-THC, THCA, CBD, CBDA, CBDV, CBDVa, CBC, CBCV, CBG, CBGV, CBGM, CBN, CBE, CBL, CBT, CBV, THCV, THCVA, THCC, and THCP.

12. The method of claim 1, wherein the one or more cannabimimetic compounds comprise alkyl amides, anandamide, guineensis, anthopogocyclolic acid, anthopogochromenic acid, and geranyl orsellinic acid.

13. The method of claim 2, wherein the plant, fungus, waste, or part thereof is selected from the genus Cannabis, Theobroma, Piper, Echinacea, Helichrysum, Rhododendron, Acmella, Rhodiola, Tuber, Copelandia, Gymnopilus, Inocybe, Panaeolus, Pholiotina, Pluteus, Psilocybe, or Hericium.

14. The method of claim 13, wherein the plant, waste, or part thereof is selected from the genus Cannabis, the predetermined ratio of acetone to water is selected from 1:2 (v/v), 1:1 (v/v), 3:2 (v/v), 2:1 (v/v), 4:1 (v/v), or 5:1 (v/v), and the one or more phytocannabinoids comprise delta-9-THC, delta-8-THC, iso-THC, THCA, CBD, CBDA, CBDV, CBDVa, CBC, CBCV, CBG, CBGV, CBGM, CBN, CBE, CBL, CBT, CBV, THCV, THCVA, THCC, and THCP.

15. The method of claim 13, wherein the plant, waste, or part thereof is selected from the genus Echinacea, the predetermined ratio of acetone to water is selected from 4:1 (v/v) to 5:1 (v/v), and the Echinacea extract comprises one or more cannabimimetic compounds, chicoric acid, echinacoside, and caftaric acid.

16. The method of claim 13, wherein the plant, waste, or part thereof is selected from the genus Rhodiola, the predetermined ratio of acetone to water is selected from 4:1 (v/v) to 5:1 (v/v), and the Rhodiola extract comprises one or more of salidroside, rosarin, and rosavin.

17. The method of claim 13, wherein the fungus, waste, or part thereof is selected from the genus Hericium, the predetermined ratio of acetone to water is selected from 4:1 (v/v) to 5:1 (v/v), and the Hericium extract comprises one or more glucans selected from alpha-glucan or beta-glucan.

18. An extract of a plant, fungus, waste, or part thereof obtained by the method of claim 1.

19. A composition comprising the extract of a plant, fungus, waste, or part thereof obtained by the method of claim 1, and at least one acceptable carrier.

20. A method of treating, preventing, or ameliorating a disease or disorder in a subject, comprising administering to the subject an effective amount of an extract of a plant, fungus, waste, or part thereof obtained by the method of claim 1, or a composition comprising the extract and at least one acceptable carrier.

21. The method of claim 20, wherein the disease or disorder comprises pain, nausea, insomnia, appetite loss, depression, inflammation, anxiety, epilepsy, seizures, cancer, multiple sclerosis, amyotrophic lateral sclerosis, Parkinson's disease, Alzheimer's disease, Crohn's disease, and post-traumatic stress disorder.
